(19) United States
(12) Patent Application Publication
(10) Pub. No.: US 2015/0023877 A1
(43) Pub. Date: Jan. 22, 2015

(54) METHODS OF PARKINSONS DISEASE DIAGNOSIS AND MONITORING TREATMENT

(71) Applicant: The Board of Trustees of the Leland Stanford Junior University, Palo Alto, CA (US)

(72) Inventors: Lihong Bu, Palo Alto, CA (US); Zhen Cheng, Mountain View, CA (US)

(21) Appl. No.: 14/333,997

(22) Filed: Jul. 17, 2014

Related U.S. Application Data
(60) Provisional application No. 61/847,729, filed on Jul. 18, 2013.

(51) Int. Cl. A61K 51/04

(52) U.S. Cl. CPC A61K 51/0455
USPC 424/1.89

(57) ABSTRACT

Embodiments of the present disclosure provide for methods of using labeled probes, methods of diagnosing Parkinson's disease and related biological events, methods of using labeled probes for monitoring and/or assessing Parkinson's disease treatment, methods of using labeled probes for diagnosing, monitoring, and/or assessing diseases with aberrant melanin-expression, and the like.

### CROSS-REFERENCE TO RELATED APPLICATION

This application claims priority to U.S. provisional application entitled “METHODS OF PARKINSON'S DISEASE DIAGNOSIS AND MONITORING TREATMENT,” having Ser. No. 61/847,729, filed on Jul. 18, 2013, which is entirely incorporated herein by reference.

### STATEMENT OF GOVERNMENT SPONSORSHIP

This invention was made with Government support under Contract/Grant No. 5R01 CA 119053, awarded by the National Institutes of Health. The Government has certain rights in this invention.

### BACKGROUND

Parkinson disease (PD) is a heterogeneous multisystem neurodegenerative disorder with a selective vulnerability of dopaminergic neurons in the substantia nigra pars compacta, which shows irreversible loss of dopamine in the striatum as a consequence. PD affects around 1% to 2% of the population over 60 years of age, and is expected to increase in prevalence as the global population ages in the twenty-first century. Thus, there is a need to find solutions to diagnosing and monitoring PD.

### SUMMARY

Embodiments of the present disclosure provide for methods of using labeled probes, methods of diagnosing Parkinson's disease and related biological events, methods of using labeled probes for monitoring and/or assessing Parkinson's disease treatment, methods of using labeled probes for diagnosing, imaging, monitoring, and/or assessing diseases with aberrant melanin-expression, and the like.

An exemplary embodiment of the present disclosure includes a method of diagnosing the presence of a Parkinson's disease in a subject, among others, that includes: administering to the subject a labeled probe, wherein the labeled probe has an affinity for neuromelanin present in the brain, wherein the amount of neuromelanin present in the brain is a biomarker for Parkinson's disease; optionally, imaging at least a portion of the brain of the subject; and detecting the labeled probe, wherein the amount of the labeled probe present in the brain corresponds with the amount of neuromelanin present in the brain, wherein the labeled probe has the following structure:

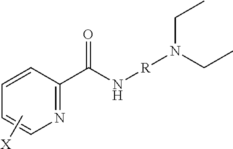

wherein X is selected from the group consisting of: ¹⁸F, ¹¹C, ¹²⁵I, ¹²⁴I, ¹³¹I, ¹²³I, ³²Cl, ³³Cl, ³⁴Cl, ⁶⁸Ga, ⁷⁴Br, ⁷⁵Br, ⁷⁶Br, ⁷⁷Br, ⁷⁸Br, ⁸⁹Zr, ¹⁸⁶Re, ¹⁸⁸Re, ⁹⁰Y, ⁸⁶Y, ¹⁷⁷Lu, and ¹⁵³Sm, wherein R is selected from the group consisting of: a substituted or unsubstituted, linear or branched, CₙH₂ₙ, where n is 1 to 12, and a substituted or unsubstituted, linear or branched, alkene group having 2 to 12 carbons, and wherein X is positioned on anyone of the carbon atoms on the heteroaromatic ring.

An exemplary embodiment of the present disclosure includes a method of monitoring the progress of a Parkinson's disease in a subject, among others, that includes: administering to the subject a labeled probe as described herein, wherein the labeled probe has an affinity for neuromelanin present in the brain, wherein the amount of neuromelanin present in the brain is a biomarker for Parkinson's disease; optionally, imaging at least a portion of the subject; detecting the labeled probe, wherein the amount of the labeled probe present in the brain corresponds to the amount of neuromelanin present in the brain; and repeating the detection of the labeled probe periodically to determine the change in the amount of neuromelanin present in the brain, wherein a change is related to the progression or regression of Parkinson's disease. In an embodiment, the method includes repeating the steps periodically to monitor the dimensions of the location corresponding to the retinal pigment epithelial (RPE) cells.

An exemplary embodiment of the present disclosure includes a method of diagnosing the presence of a Parkinson's disease in a subject, among others, that includes: administering to the subject a labeled probe as described herein, wherein the subject has been administered retinal pigment epithelial cells, wherein the labeled probe has an affinity for retinal pigment epithelial cells; optionally, imaging at least a portion of the subject; and detecting the labeled probe, wherein the location of the labeled probe corresponds to the location of the retinal pigment epithelial cells.

An exemplary embodiment of the present disclosure includes a method of monitoring the progress of a Parkinson's disease in a subject, among others, that includes: administering to the subject a labeled probe, wherein the subject has been administered retinal pigment epithelial cells, wherein the labeled probe has an affinity for retinal pigment epithelial (RPE) cells (e.g., porcine retinal pigment epithelial (pRPE) cells); optionally, imaging at least a portion of the subject; and detecting the labeled probe, wherein the location of the labeled probe corresponds to the location of the retinal pigment epithelial cells, wherein the dimensions of the location is monitored over time.

### BRIEF DESCRIPTION OF THE DRAWINGS

Further aspects of the present disclosure will be more readily appreciated upon review of the detailed description of its various embodiments, described below, when taken in conjunction with the accompanying drawings.

FIG. 1 illustrates a schematic drawing of ¹⁸F-P3BZA for imaging of pRPE cells in rat brains. The melanotic pRPE cells (shown in black in Eppendorf tube in the Figure) attached to gelatin microcarriers (pRPE-GMs) as the source of donor cells and amelanotic ARPE-19 cells (shown in white in Eppendorf tube in the Figure) attached to GMs (APRE-19-GMs) or GMs alone as controls, were intrastriatal implanted (left or right striatum, respectively) into normal rats. The small lipophilic melanin targeted probe, ¹⁸F-P3BZA, could serve as a valid PET probe for monitoring and trafficking the pRPE cells after their intrastriatal transplantation. Arrows indicate the striatum, dopaminergic neurons, dopamine receptor and pRPE-GMs.

FIGS. 2A-2D illustrates in vitro cell uptake of ¹⁸F-P3BZA and quantification analysis of melanin. FIG. 2A illustrates a bar chart that shows time dependent uptake of ¹⁸F-P3BZA after incubation for 15, 30, 60, or 120 minutes in pRPE cells and control ARPE19 cells, with or without L-tyrosine pretreatment for 24 h. Results are expressed as percentage of total applied radioactivity and are mean of triplicate measurements±standard deviations. There are significant differences between groups with and without L-tyrosine treatment. (*, P<0.05). There are significant differences between pRPE cell groups (without L-tyrosine treatment) and control ARPE-19 cell groups (#, P<0.05;). FIG. 2B illustrates a bar chart that shows the uptake of ¹⁸F-P3BZA after 30 min incubation in pRPE cells of different passages numbers and control ARPE19 cells, with or without L-tyrosine pretreatment. Results are expressed as percentage of total applied radioactivity and are mean of triplicate measurements±standard deviations. There are significant differences between pRPE-P8 group (without L-tyrosine treatment) and control ARPE-19 cell groups (*, P<0.05). There are significant differences between pRPE-P5 group and pRPE-P8 group (*, P<0.05) without L-tyrosine treatment. There are significant differences between pRPE-P3 group and pRPE-P5 group (*, P<0.05) without L-tyrosine treatment. There are significant differences between groups with or without L-tyrosine treatment (*, P<0.05). FIG. 2C is a graph that shows correlation between uptake of ¹⁸F-P3BZA and melanin content at different passage numbers of pRPE cells and control ARPE-19 cells. FIG. 2D illustrates a bar chart that shows results of quantification analysis of melanin for pRPE cells of different passage numbers and control ARPE-19 cells with or without tyrosine incubation for 24 hours (n=4). Data are means±standard deviations (SD).

FIGS. 3A and 3B illustrate the in vivo dynamic PET imaging of ¹⁸F-P3BZA in normal rat brain. FIG. 3A illustrates representative decay-corrected coronal images of normal rats brain. Yellow arrow shows the brain PET/CT image at 28 min after injection of ¹⁸F-P3BZA. FIG. 3B illustrates a time-activity curves derived from PET studies in normal rat brain across the striatum level. Results are expressed as percentage of injected dose per gram brain tissue±standard deviation (% ID/g±SD, n=4).

FIGS. 4A-4E illustrate in vivo static PET/CT images of ¹⁸F-P3BZA at 2 days after cell implantation. FIG. 4A shows the representative decay-corrected coronal PET/CT images after implantation of gelatin microcarrier-bound pRPE cells (red arrow). Gelatin microcarrier-bound ARPE-19 cells (green arrow) and gelatin microcarrier alone (yellow arrows) were used as controls. FIG. 4B illustrates a bar chart that shows results of quantitative analysis of small animal coronal PET images for uptake of ¹⁸F-P3BZA. The signal of gelatin microcarrier-bound pRPE cells (pRPE-GMs) is much higher than that of gelatin microcarrier-bound ARPE-19 cells or gelatin microcarriers alone. Results are expressed as percentage of ID per gram of brain tissue±standard deviation. There were four animals in each group. FIG. 4C illustrates a bar chart that shows gelatin microcarrier-bound RPE cell-to-gelatin microcarrier uptake ratios for gelatin microcarrier-bound pRPE cells (pRPE-GMs) and gelatin microcarrier-bound ARPE-19 cells (ARPE-GMs). FIG. 4D illustrate a representative hematoxylin-eosin (H&E)-stained and coronal autoradiography images. The combination of autoradiography and hematoxylin-eosin staining further demonstrates that ¹⁸F-P3BZA uptake in gelatin microcarrier-bound pRPE cells (pRPE-GMs) is much higher than that in either gelatin microcarrier-bound ARPE-19 cells (ARPE-19-GMs) or gelatin microcarriers (GMs) alone. Green circles=implantation sites for gelatin microcarriers, red circles=implantation sites for gelatin microcarrier-bound pRPE cells, yellow circles=implantation sites for gelatin microcarrier-bound ARPE-19 cells. FIG. 4E, Bar chart shows results of quantitative analysis of autoradiography images processed with Image J software. pRPE-GMs=gelatin microcarrier-bound pRPE cells, GMs=gelatin microcarriers, ARPE-19-GMs=gelatin microcarrier-bound ARPE-19 cells. Results of radioactivity in autoradiography images are expressed as brightness in circle. Value of brightness is generated with Image J software. U.A.=arbitrary units.

FIGS. 5A-5B illustrate longitudinal ¹⁸F-P3BZA PET/CT imaging 2, 9, and 16 days after implantation of gelatin microcarrier-bound pRPE cells (pRPE-GMs) in normal rats. FIG. 5A illustrates a representative decay-corrected longitudinal PET/CT images. Red arrows=implantation sites for gelatin microcarrier-bound pRPE cells, yellow arrows=implantation sites for gelatin microcarriers (GMs). FIG. 5B illustrates a bar chart that shows results of quantification analysis of longitudinal ¹⁸F-P3BZA PET/CT. Results are means±standard deviations. Four rats were evaluated at each time point.

FIG. 6 illustrates representative photomicrographs of Fontana-Masson and immunohistochemistry staining of rat brain sections. The rat brain intrastriatal implanted with the control GMs and pRPE-GMs were sacrificed on day 2, 9 and 16 post-implantation and the brain tissues were sliced and stained. By immunohistochemistry staining, pRPE cells were characterized as RPE-65 positive and adhered onto the surface of GMs. Fontana-Masson staining identified melanin as black pigmentations in granular or cluster shapes. On FMS images, nucleus was stained red by nuclear fast red. Green arrow indicates the representative pRPE cells with matched melanin presented inside. Overview images taken at 10× magnification with a scale bar that represents 100 nm. Panels with dashed borders are zoomed (14× magnification) and cropped sections from the overview panels with a scale bar that represents 25 μm (cropped regions are indicated by the small dashed rectangle in the overview panels).

FIG. 7A illustrates the basic chemical structure in this disclosure.

FIG. 7B illustrates a synthetic scheme for preparation of cold N-(2-(diethylamino)ethyl)-¹⁹F-5-fluoropicolinamide and hot N-(2-(diethylamino)ethyl)-¹⁸F-5-fluoropicolinamide.

### DETAILED DESCRIPTION

Before the present disclosure is described in greater detail, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit (unless the context clearly dictates otherwise), between the upper and lower limit of that range, and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of chemistry, synthetic organic chemistry, biochemistry, biology, molecular biology, molecular imaging, and the like, which are within the skill of the art. Such techniques are explained fully in the literature.

The following examples are put forth so as to provide those of ordinary skill in the art with a complete disclosure and description of how to perform the methods and use the compositions and compounds disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

It must be noted that, as used in the specification and the appended claims, the singular forms “a,” “an,” and “the” include plural referents unless the context clearly dictates otherwise. Thus, for example, reference to “a support” includes a plurality of supports. In this specification and in the claims that follow, reference will be made to a number of terms that shall be defined to have the following meanings unless a contrary intention is apparent.

### DEFINITIONS

In describing and claiming the disclosed subject matter, the following terminology will be used in accordance with the definitions set forth below.

The term “substituted” refers to any one or more hydrogens on the designated atom that can be replaced with a selection from the indicated group, provided that the designated atom's normal valence is not exceeded. The term “substituted,” can mean that the substituted group may contain in place of one or more hydrogens a group such as alkyl (e.g., C1 to C6), hydroxy, amino, halo, trifluoromethyl, cyano, —NH(alkyl), —N(alkyl)₂, lower (e.g., C1 to C6) alkoxy, lower alkylthio, or carboxy, and thus embraces the terms haloalkyl, alkoxy, fluorobenzyl, and the sulfur and phosphorous containing substitutions referred to below.

By “administration” or “administering” is meant introducing a probe or a labeled probe (also referred to as the “imaging agent”) of the present disclosure into a subject. The preferred route of administration of the compounds is intravenous. However, any route of administration, such as oral, topical, subcutaneous, peritoneal, intraarterial, inhalation, vaginal, rectal, nasal, introduction into the cerebrospinal fluid, or instillation into body compartments can be used.

In accordance with the present disclosure, “a detectably effective amount” of the probe of the present disclosure is defined as an amount sufficient to yield an acceptable image using equipment that is available for clinical use. A detectably effective amount of the probe of the present disclosure may be administered in more than one injection. The detectably effective amount of the probe of the present disclosure can vary according to factors such as the degree of susceptibility of the individual, the age, sex, and weight of the individual, idiosyncratic responses of the individual, and the like. Detectably effective amounts of the probe of the present disclosure can also vary according to instrument and film-related factors. Optimization of such factors is well within the level of skill in the art.

As used herein, the term “host” or “subject” includes humans, mammals (e.g., cats, dogs, horses, etc.), and poultry. Typical hosts to which embodiments of the present disclosure may be administered will be mammals, particularly primates, especially humans. For veterinary applications, a wide variety of subjects will be suitable, e.g., livestock such as cattle, sheep, goats, cows, swine, and the like; poultry such as chickens, ducks, geese, turkeys, and the like; and domesticated animals particularly pets such as dogs and cats. For diagnostic or research applications, a wide variety of mammals will be suitable subjects, including rodents (e.g., mice, rats, hamsters), rabbits, primates, and swine such as inbred pigs and the like. Additionally, for in vitro applications, such as in vitro diagnostic and research applications, body fluids and cell samples of the above subjects will be suitable for use, such as mammalian (particularly primate such as human) blood, urine, or tissue samples, or blood, urine, or tissue samples of the animals mentioned for veterinary applications. In some embodiments, a system includes a sample and a host. The term “living subject” refers to a subject noted above that is alive and is not dead. The term “living subject” refers to the entire subject and not just a part excised (e.g., a liver or other organ) from the living subject.

The term “sample” can refer to a tissue sample, cell sample, a fluid sample, and the like. The sample may be taken from a subject. The tissue sample can include hair (including roots), buccal swabs, blood, saliva, semen, muscle, or from any internal organs. The fluid may be, but is not limited to, urine, blood, ascites, pleural fluid, spinal fluid, and the like. The body tissue can include, but is not limited to, skin, muscle, endometrial, uterine, and cervical tissue. In the present disclosure, the source of the sample is not critical.

The term “detectable” refers to the ability to detect a signal over the background signal.

The term “detectable signal” is a signal derived from non-invasive imaging techniques such as, but not limited to, positron emission tomography (PET) or single photon emission computed tomography (SPECT). The detectable signal is detectable and distinguishable from other background signals that may be generated from the subject. In other words, there is a measurable and statistically significant difference (e.g., a statistically significant difference is enough of a difference to distinguish among the detectable signal and the background, such as about 0.1%, 1%, 3%, 5%, 10%, 15%, 20%, 25%, 30%, or 40% or more difference between the detectable signal and the background) between the detectable signal and the background. Standards and/or calibration curves can be used to determine the relative intensity of the detectable signal and/or the background.

### General Discussion

Embodiments of the present disclosure provide for methods of using labeled probes, methods of diagnosing Parkinson's disease and related biological events, methods of using labeled probes for monitoring and/or assessing Parkinson's disease treatment, methods of using labeled probes for diagnosing, monitoring, and/or assessing diseases with aberrant melanin-expression, and the like. In particular, the present disclosure includes methods relating to non-invasive imaging (e.g., using positron emission tomography (PET) imaging system) using labeled probes, such as a 3-¹⁸F-fluoropicolinamide probe, in vivo, for diagnosing Parkinson's disease and for monitoring and/or accessing Parkinson's disease treatment by monitoring intrastriatal implanted retinal pigment epithelial cells.

Portions of the present disclosure discuss labeled probes while other portions describe a specific embodiment of the labeled probes, the 3-¹⁸F-fluoropicolinamide probe. Discussions focusing on the 3-¹⁸F-fluoropicolinamide probe are not limiting to the scope of the disclosure, rather those discussions are merely describing an exemplary embodiment of the present disclosure.

Embodiments of the present disclosure include methods for imaging a sample (e.g., tissue or cell(s)) or a subject, that includes contacting a sample with or administering to a subject a labeled probe (a 3-¹⁸F-fluoropicolinamide probe) and imaging with a PET imaging system. The imaging can be performed in vivo and/or in vitro.

Neuromelanin is a biomarker for Parkinson's disease. It has been determined that the labeled probes can pass through the blood brain barrier and accumulate in the brain. In a particular embodiment, labeled probes can be used to image Parkinson's disease by monitoring the change in the amount of neuromelanin present in the brain. A change in the amount of neuromelanin present in the brain can be used to detect the presence of Parkinson's disease in a subject and/or monitor the progression of Parkinson's disease in a subject. When the amount of neuromelanin present in the brain decreases, the subject may be in the early stages of Parkinson's disease. The relative amount and/or the decrease will depend upon the individual, stage of disease, and the like. Detection of Parkinson's disease at an early stage allows for early intervention, which can result in better long term treatment results. Detecting the change of the amount of neuromelanin present in the brain over a time period can be used to determine the progression of Parkinson's disease and used to access if the treatment is effective or not and if the treatment should be adjust or changed.

In an embodiment, the labeled probes can be used to monitor and/or access Parkinson's disease treatment with retinal pigment epithelial cells, specifically Human retinal pigment epithelial (hRPE) cells. Human retinal pigment epithelial cells have been studied as a source of donor cells for neural transplantation in the treatment of Parkinson's disease. In order to understand the anti-Parkinson's disease treatment efficacy of hRPE cell implantation, the activities of hRPE cells can be imaged after implantation in vivo. The labeled probe is able to identify and bind (e.g., have an affinity for) to melanotic porcine RPE (pRPE) cells specifically and effectively in vitro and in vivo. Thus, the labeled probes can be used for monitoring the intrastriatal transplantation of RPE cells. These findings can be used to improve the efficacy of hRPE cell therapy for Parkinson's disease.

In an exemplary embodiment, the labeled probes can also be used to diagnose other diseases with aberrant melanin-expression, including melanoma and catecholamine-secreting pheochromocytoma, and other neurodegenerative disorders, such as Alzheimer's disease and Down's syndrome with neuromelanin loss in the locus coeruleus. In addition, the labeled probe can be used to monitor the response to therapy for each of these.

In an embodiment, the 3-¹⁸F-fluoropicolinamide probe can be imaged or detected using imaging systems such as positron emission tomography (PET) imaging systems, single photon emission computed tomography (SPECT), and the like. In an embodiment, PET imaging is a preferred embodiment. Other types of labeled probes can use appropriate imaging systems.

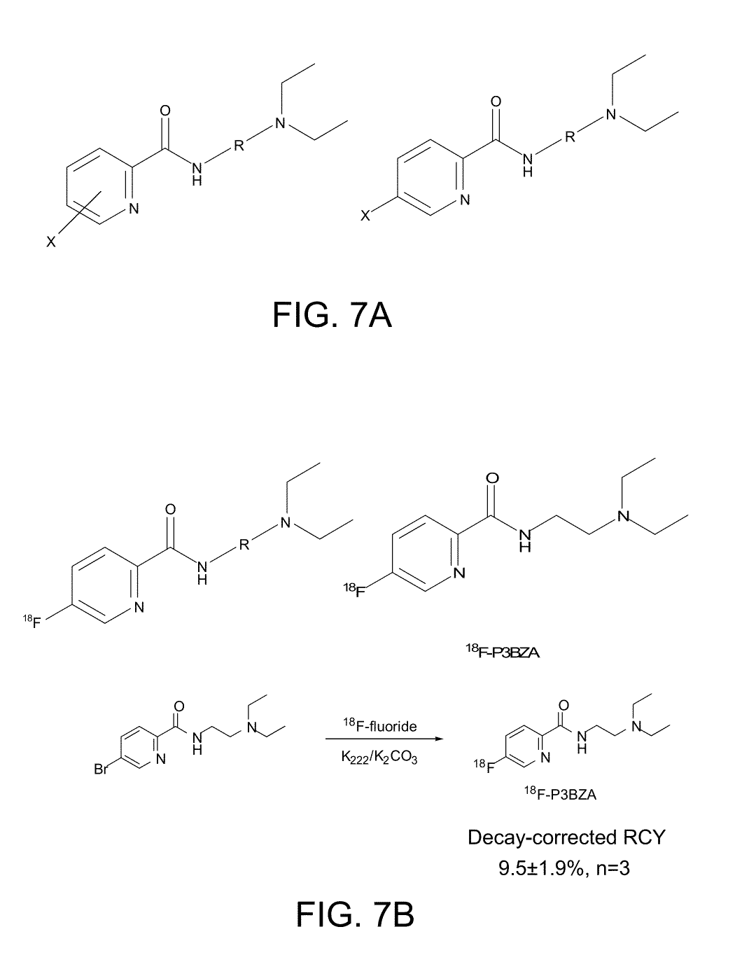

FIG. 7A illustrates labeled probes that can be used in diagnosing, imaging, localizing, monitoring, and/or assessing Parkinson's disease and Parkinson's disease treatment, or related biological events. In particular, the present disclosure includes methods relating to non-invasive imaging (e.g., using positron emission tomography (PET) imaging system) using the labeled probe in vivo. In an exemplary embodiment X can be a radiolabel such as ¹⁸F, ¹¹C, ¹²⁵I, ¹²⁴I, ¹³¹I, ¹²³I, ³²Cl, ³³Cl, ³⁴Cl, ⁶⁸Ga, ⁷⁴Br, ⁷⁵Br, ⁷⁶Br, ⁷⁷Br, ⁷⁸Br, ⁸⁹Zr, ¹⁸⁶Re, ¹⁸⁸Re, ⁹⁰Y, ⁸⁶Y, ¹⁷⁷Lu, or ¹⁵³Sm. In an embodiment, the radiolabel, X, can be ¹⁸F, ⁷⁶Br, or ¹²³I, ¹²⁴I or ¹³¹I, which are suitable for use in peripheral medical facilities and PET clinics. In other embodiments, the radiolabel or PET isotope, X, can include, but is not limited to, ⁶⁴Cu, ¹²⁴I, ^{76/77}Br, ⁸⁶Y, ⁸⁹Zr, or ⁶⁸Ga. In an exemplary embodiment R can be an alkyl group such as CₙH₂ₙ, where n can be 1 to 12, that can be substituted or unsubstituted and/or linear or branched, or an alkene group (e.g., 1 to 12 carbons with one or more double bonds that can be substituted or unsubstituted and/or linear or branched).

FIG. 7B illustrates embodiments of the labeled probe and a method of making a specific labeled probe. R can be a group as defined in reference to FIG. 7A. Specifically, FIG. 7B illustrates an embodiment of the 3-¹⁸F-fluoropicolinamide probe and a method of making the 3-¹⁸F-fluoropicolinamide probe.

In an embodiment, the 3-¹⁸F-fluoropicolinamide probe includes a label, ¹⁸F, that can be used to detect, image, or otherwise identify the 3-¹⁸F-fluoropicolinamide probe, quantify the amount of 3-¹⁸F-fluoropicolinamide probe, determine the location of the 3-¹⁸F-fluoropicolinamide probe (e.g., in imaging), and combinations thereof. Fluorine-18 (t_{1/2}=109.7 min; β⁺, 99%) is an ideal short-lived PET isotope for labeling small molecules. Additional details regarding the 3-¹⁸F-fluoropicolinamide probe are described in Example 1.

In an embodiment and as described in Example 1, the melanin targeted positron emission tomography (PET) probe, N-(2-(diethylamino)ethyl)-¹⁸F-5-fluoropicolinamide (¹⁸F-P3BZA), shows high accumulation in porcine RPE (pRPE) cells (10.72±0.38% of applied activity within 30 minutes after co-incubation) and excellent imaging contrast (evidenced by much higher pRPE-gelatin microcarriers to control gelatin microcarriers uptake ratio than ARPE-19-gelatin microcarriers to gelatin microcarriers uptake ratio (3.05±0.57 vs. 1.21±0.36, p=0.04)), and can be used to longitudinal monitor intrastriatal transplanted RPE cells in living subjects.

In addition, ¹⁸F-P3BZA identifies and binds to transplanted porcine RPE cells specifically in a melanin-depended manner as evident by the high correlation (R²=0.91) between the ¹⁸F-P3BZA uptake and the melanin content in porcine RPE cells at different passages, and the consistency between the longitudinal PET imaging results and the corresponding postmortem analysis. In vivo ¹⁸F-P3BZA PET reveals that the activity of implanted porcine RPE cells decreases over time following their implantation into the rat brain. ¹⁸F-P3BZA shows favorable pharmacokinetics in normal rat brain (the uptakes of ¹⁸F-P3BZA in rat brain are 7.61±0.30, 3.54±0.21, and 1.04±0.37% ID/g at 2, 28 and 60 min after injection, respectively), which makes it suitable for targeted imaging of RPE cells in brain.

### Methods of Use

Embodiments of this disclosure include, but are not limited to: methods of detecting the labeled probe (e.g., 3-¹⁸F-fluoropicolinamide probe), methods of imaging a sample or a subject using the labeled probe (e.g., 3-¹⁸F-fluoropicolinamide probe), methods of imaging Parkinson's disease (e.g., presence or progression) or related biological events, methods of monitoring or accessing Parkinson's disease treatment; diseases with aberrant melanin-expression; and the like.

Embodiments of the present disclosure can be used to image, detect, study, monitor, evaluate, assess, and/or screen, Parkinson's disease, Parkinson's disease treatment, diseases with aberrant melanin-expression, Parkinson's disease agents, in vivo or in vitro using the labeled probe (e.g., 3-¹⁸F-fluoropicolinamide probe). Although the methods describe below are directed to Parkinson's disease, the methods can be applied to other diseases with aberrant melanin-expression.

In a particular embodiment, the 3-¹⁸F-fluoropicolinamide probe can be used in imaging Parkinson's disease and determine its presence and/or progression. For example, the 3-¹⁸F-fluoropicolinamide probe is provided or administered to a subject in an amount effective to result in uptake of the 3-¹⁸F-fluoropicolinamide probe. The subject is then introduced to an appropriate imaging system (e.g., PET system) for a certain amount of time (e.g., this depends on radioisotope being used, for ¹⁸F, it usually could be up to 4 hours). The 3-¹⁸F-fluoropicolinamide probe can pass the blood barrier in the brain, associate with neuormelanin and, can be detected using the imaging system. The detected signal from the 3-¹⁸F-fluoropicolinamide probe can be correlated to the amount of the 3-¹⁸F-fluoropicolinamide probe in the brain, which correlates with the amount of neuromelanin in the brain. In an embodiment, the detected signal can be used to form an image (e.g., image of the brain or portions thereof). As a result, the onset of Parkinson's disease can be determined by the amount of or the change in the amount of neuromelanin in the brain. Other labeled probes can be used in a similar manner.

In an embodiment, the steps of this method can be repeated at determined intervals so the presence/absence/progress of the disease can be monitored as a function of time and/or treatment. In particular, the 3-¹⁸F-fluoropicolinamide probe can find use in a host undergoing treatment (e.g., using a drug), to aid in imaging (e.g., visualizing) the response of the disease to the treatment. In this embodiment, the 3-¹⁸F-fluoropicolinamide probe is typically visualized and sized prior to treatment, and periodically (e.g., daily, weekly, monthly, intervals in between these, and the like) during treatment, and the like, to monitor the progression of the disease. Other labeled probes can be used in a similar manner.

In a particular embodiment, the 3-¹⁸F-fluoropicolinamide probe can be used in monitoring and/or accessing Parkinson's disease treatment. For example, the 3-¹⁸F-fluoropicolinamide probe is provided or administered to a subject in an amount effective to result in uptake of the 3-¹⁸F-fluoropicolinamide probe. The subject is provided or administered retinal pigment epithelial cells (e.g., Human retinal pigment epithelial cells) to treat Parkinson's disease. The subject is then introduced to an appropriate imaging system (e.g., PET system) for a certain amount of time (e.g., this depends on radioisotope being used, for ¹⁸F, it usually could be up to 4 hours). The 3-¹⁸F-fluoropicolinamide probe can pass the blood barrier in the brain, associate with the retinal pigment epithelial cells and, can be detected using the imaging system. The detected signal from the 3-¹⁸F-fluoropicolinamide probe can be correlated to the amount of the 3-¹⁸F-fluoropicolinamide probe in the brain, which correlates with the location and amount of retinal pigment epithelial cells in the brain. In an embodiment, the steps of this method can be repeated at determined intervals so the progression or regression of the disease can be monitored as a function of time and/or treatment. As a result, the progression and effectiveness of the treatment of Parkinson's disease can be determined. Other labeled probes can be used in a similar manner.

Embodiments of the 3-¹⁸F-fluoropicolinamide probe also find use as a screening tool in vitro to select compounds for use in treating Parkinson's disease. The disease could be monitored by incubating the cells with the disease with the 3-¹⁸F-fluoropicolinamide probe during or after incubation with one or more candidate drugs. The ability of the drug compound to affect the disease can be imaged over time using the 3-¹⁸F-fluoropicolinamide probe. Other labeled probes can be used in a similar manner.

It should be noted that the amount effective to result in uptake of the labeled probe (e.g., 3-¹⁸F-fluoropicolinamide probe) into the subject (brain) may depend upon a variety of factors, including for example, the age, body weight, general health, sex, and diet of the host; the time of administration; the route of administration; the rate of excretion of the specific probe employed; the duration of the treatment; the existence of other drugs used in combination or coincidental with the specific composition employed; and like factors well known in the medical arts.

### Kits

The present disclosure also provides packaged compositions or pharmaceutical compositions comprising a pharmaceutically acceptable carrier and a labeled probe (e.g., 3-¹⁸F-fluoropicolinamide probe) of the disclosure. In certain embodiments, the packaged compositions or pharmaceutical composition includes the reaction precursors to be used to generate the labeled probe according to the present disclosure. Other packaged compositions or pharmaceutical compositions provided by the present disclosure further include indicia including at least one of: instructions for using the labeled probe to image a host, or host samples (e.g., cells or tissues), which can be used as an indicator of conditions including, but not limited to, Parkinson's disease and biological related events.

Embodiments of this disclosure encompass kits that include, but are not limited to, the labeled probe (e.g., 3-¹⁸F-fluoropicolinamide probe) and directions (written instructions for their use). The kit can further include appropriate buffers and reagents known in the art for administering various combinations of the components listed above to the host cell or host organism. The imaging agent and carrier may be provided in solution or in lyophilized form. When the imaging agent and carrier of the kit are in lyophilized form, the kit may optionally contain a sterile and physiologically acceptable reconstitution medium such as water, saline, buffered saline, and the like.

### Dosage Forms

Embodiments of the present disclosure can be included in one or more of the dosage forms mentioned herein. Unit dosage forms of the pharmaceutical compositions (the “composition” includes at least the labeled probe, e.g., 3-¹⁸F-fluoropicolinamide probe) of this disclosure may be suitable for oral, mucosal (e.g., nasal, sublingual, vaginal, buccal, or rectal), parenteral (e.g., intramuscular, subcutaneous, intravenous, intra-arterial, or bolus injection), topical, or transdermal administration to a patient. Examples of dosage forms include, but are not limited to: tablets; caplets; capsules, such as hard gelatin capsules and soft elastic gelatin capsules; cachets; troches; lozenges; dispersions; suppositories; ointments; cataplasms (poultices); pastes; powders; dressings; creams; plasters; solutions; patches; aerosols (e.g., nasal sprays or inhalers); gels; liquid dosage forms suitable for oral or mucosal administration to a patient, including suspensions (e.g., aqueous or non-aqueous liquid suspensions, oil-in-water emulsions, or water-in-oil liquid emulsions), solutions, and elixirs; liquid dosage forms suitable for parenteral administration to a patient; and sterile solids (e.g., crystalline or amorphous solids) that can be reconstituted to provide liquid dosage forms suitable for parenteral administration to a patient.

The composition, shape, and type of dosage forms of the compositions of the disclosure typically vary depending on their use. For example, a parenteral dosage form may contain smaller amounts of the active ingredient than an oral dosage form used to treat the same condition or disorder. These and other ways in which specific dosage forms encompassed by this disclosure vary from one another will be readily apparent to those skilled in the art (See, e.g., Remington's Pharmaceutical Sciences, 18th ed., Mack Publishing, Easton, Pa. (1990)).

Typical compositions and dosage forms of the compositions of the disclosure can include one or more excipients. Suitable excipients are well known to those skilled in the art of pharmacy or pharmaceutics, and non-limiting examples of suitable excipients are provided herein. Whether a particular excipient is suitable for incorporation into a composition or dosage form depends on a variety of factors well known in the art including, but not limited to, the way in which the dosage form will be administered to a patient. For example, oral dosage forms, such as tablets or capsules, may contain excipients not suited for use in parenteral dosage forms. The suitability of a particular excipient may also depend on the specific active ingredients in the dosage form. For example, the decomposition of some active ingredients can be accelerated by some excipients, such as lactose, or by exposure to water. Active ingredients that include primary or secondary amines are particularly susceptible to such accelerated decomposition.

The disclosure encompasses compositions and dosage forms of the compositions of the disclosure that can include one or more compounds that reduce the rate by which an active ingredient will decompose. Such compounds, which are referred to herein as “stabilizers,” include, but are not limited to, antioxidants such as ascorbic acid, pH buffers, or salt buffers. In addition, pharmaceutical compositions or dosage forms of the disclosure may contain one or more solubility modulators, such as sodium chloride, sodium sulfate, sodium or potassium phosphate, or organic acids. An exemplary solubility modulator is tartaric acid.

“Pharmaceutically acceptable salt” refers to those salts that retain the biological effectiveness and properties of the free bases and that are obtained by reaction with inorganic or organic acids such as hydrochloric acid, hydrobromic acid, sulfuric acid, nitric acid, phosphoric acid, methanesulfonic acid, ethanesulfonic acid, p-toluenesulfonic acid, salicylic acid, malic acid, maleic acid, succinic acid, tartaric acid, citric acid, and the like.

Embodiments of the present disclosure include pharmaceutical compositions that include the labeled probe (e.g., 3-¹⁸F-fluoropicolinamide probe), pharmaceutically acceptable salts thereof, with other chemical components, such as physiologically acceptable carriers and excipients. One purpose of a pharmaceutical composition is to facilitate administration of labeled probe (e.g., 3-¹⁸F-fluoropicolinamide probe) to a subject (e.g., human).

Embodiments of the present disclosure may be salts and these salts are within the scope of the present disclosure. Reference to a compound of any of the formulas herein is understood to include reference to salts thereof, unless otherwise indicated. The term “salt(s)”, as employed herein, denotes acidic and/or basic salts formed with inorganic and/or organic acids and bases. In addition, when an embodiment of the present disclosure contains both a basic moiety and an acidic moiety, zwitterions (“inner salts”) may be formed and are included within the term “salt(s)” as used herein. Pharmaceutically acceptable (e.g., non-toxic, physiologically acceptable) salts are preferred, although other salts are also useful, e.g., in isolation or purification steps, which may be employed during preparation. Salts of the compounds of an active compound may be formed, for example, by reacting an active compound with an amount of acid or base, such as an equivalent amount, in a medium such as one in which the salt precipitates or in an aqueous medium followed by lyophilization.

Embodiments of the present disclosure that contain a basic moiety may form salts with a variety of organic and inorganic acids. Exemplary acid addition salts include acetates (such as those formed with acetic acid or trihaloacetic acid, for example, trifluoroacetic acid), adipates, alginates, ascorbates, aspartates, benzoates, benzenesulfonates, bisulfates, borates, butyrates, citrates, camphorates, camphorsulfonates, cyclopentanepropionates, digluconates, dodecylsulfates, ethanesulfonates, fumarates, glucoheptanoates, glycerophosphates, hemisulfates, heptanoates, hexanoates, hydrochlorides (formed with hydrochloric acid), hydrobromides (formed with hydrogen bromide), hydroiodides, 2-hydroxyethanesulfonates, lactates, maleates (formed with maleic acid), methanesulfonates (formed with methanesulfonic acid), 2-naphthalenesulfonates, nicotinates, nitrates, oxalates, pectinates, persulfates, 3-phenylpropionates, phosphates, picrates, pivalates, propionates, salicylates, succinates, sulfates (such as those formed with sulfuric acid), sulfonates (such as those mentioned herein), tartrates, thiocyanates, toluenesulfonates such as tosylates, undecanoates, and the like.

Embodiments of the present disclosure that contain an acidic moiety may form salts with a variety of organic and inorganic bases. Exemplary basic salts include ammonium salts, alkali metal salts such as sodium, lithium, and potassium salts, alkaline earth metal salts such as calcium and magnesium salts, salts with organic bases (for example, organic amines) such as benzathines, dicyclohexylamines, hydrabamines (formed with N,N-bis(dehydroabietyl)ethylenediamine), N-methyl-D-glucamines, N-methyl-D-glucamides, t-butyl amines, and salts with amino acids such as arginine, lysine, and the like.

Basic nitrogen-containing groups may be quaternized with agents such as lower alkyl halides (e.g., methyl, ethyl, propyl, and butyl chlorides, bromides and iodides), dialkyl sulfates (e.g., dimethyl, diethyl, dibutyl, and diamyl sulfates), long chain halides (e.g., decyl, lauryl, myristyl and stearyl chlorides, bromides and iodides), aralkyl halides (e.g., benzyl and phenethyl bromides), and others.

Solvates of the compounds of the disclosure are also contemplated herein. Solvates of the compounds are preferably hydrates.

The amounts and a specific type of active ingredient (e.g., a labeled probe such as 3-¹⁸F-fluoropicolinamide probe) in a dosage form may differ depending on various factors. It will be understood, however, that the total daily usage of the compositions of the present disclosure will be decided by the attending physician or other attending professional within the scope of sound medical judgment. The specific effective dose level for any particular host will depend upon a variety of factors, including for example, the activity of the specific composition employed; the specific composition employed; the age, body weight, general health, sex, and diet of the host; the time of administration; the route of administration; the rate of excretion of the specific compound employed; the duration of the treatment; the existence of other drugs used in combination or coincidental with the specific composition employed; and like factors well known in the medical arts. For example, it is well within the skill of the art to start doses of the composition at levels lower than those required to achieve the desired effect and to gradually increase the dosage until the desired effect is achieved.

### EXAMPLES

Now having described the embodiments of the disclosure, in general, the examples describe some additional embodiments. While embodiments of the present disclosure are described in connection with the example and the corresponding text and figures, there is no intent to limit embodiments of the disclosure to these descriptions. On the contrary, the intent is to cover all alternatives, modifications, and equivalents included within the spirit and scope of embodiments of the present disclosure.

### Example 1

### In Vivo Longitudinal Molecular Imaging of Intrastriatal Transplanted RPE Cells by ¹⁸F-P3BZA PET/CT

### Brief Introduction:

Human retinal pigment epithelial (hRPE) cells have been studied as a source of donor cells for neural transplantation in the treatment of Parkinson disease (PD). In order to understand the anti-PD treatment efficacy of hRPE cell implantation, it is critical to develop a technique for imaging the activities of hRPE cells following their implantation in vivo. Because RPE cells have a high melanin content, we hypothesized that the small melanin targeted probe, N-(2-(diethylamino)ethyl)-¹⁸F-5-fluoropicolinamide (¹⁸F-P3BZA), could serve as a valid positron emission tomography (PET) probe for monitoring intrastriatal transplantation of RPE cells. Our studies demonstrated that ¹⁸F-P3BZA was able to identify and bind to melanotic porcine RPE (pRPE) cells specifically and effectively in vitro and in vivo. Furthermore, longitudinal PET/CT scan revealed that the activity of implanted pRPE cells decreased following their implantation into the host, as evidenced by a significant reduction in uptake of ¹⁸F-P3BZA by the pRPE cells. Autoradiography, H&E and Fontana-Masson staining confirmed the in vivo imaging results. In conclusion, ¹⁸F-P3BZA PET/CT provides unprecedented opportunities to visualize and detect the activity of implanted RPE cells in vivo. Our findings may contribute to improved efficacy of hRPE cell therapy for PD.

### Introduction:

Parkinson disease (PD) is a heterogeneous multisystem neurodegenerative disorder with a selective vulnerability of dopaminergic neurons in the substantia nigra pars compacta, which shows irreversible loss of dopamine in the striatum as a consequence. PD affects around 1% to 2% of the population over 60 years of age, and is expected to increase in prevalence as the global population ages in the twenty-first century¹. Substantial research effort has been focused on developing new and effective regimens to diagnose and treat PD. Striatal transplantation of cells which can produce dopamine (DA) or L-DOPA (L-3,4-dihydroxyphenylalanine, levodopa) has been actively investigated for PD treatment in preclinical and clinical trials. These treatment regimens may provide a long-term, continuous dopaminergic source and avoid motor complications because of the fluctuations and chronic intermittent dopaminergic stimulation associated with oral levodopa administration²⁻⁵.

Retinal pigment epithelial (RPE) cells accumulate L-DOPA during eye development as a precursor for the formation of their characteristic melanin pigment⁶. Apart from L-DOPA production, human RPE (hRPE) cells can also produce a small amount of DA directly and possess other molecular machinery involved in DA production and regulation⁷⁻¹⁰. Therefore, hRPE cells attached to gelatin microcarriers (GMs) have been extensively explored as a promising source of donor cells for neural transplantation in the treatment of PD by providing “L-DOPA pumps” to maintain stable L-DOPA levels. Furthermore, attachment to gelatin microcarriers to produce hRPE-GM appears to grant a protective advantage and negates the need for immunosuppression¹¹. Numerous preclinical studies in rodents and non-human primates indicate that hRPE-GMs implanted into the striatum can significantly ameliorate the behavioral deficits of the recipients^{12,13}. Moreover, several clinical trials suggest that hRPE-GMs grafts can improve the motor and quality of life measurements in some patients, though outcomes are highly variable¹⁴⁻¹⁶. For example, in a pilot clinical trial (6 patients), a 48% reduction in Unified Parkinson's Disease Rating Scale motor score was achieved at 12 months and up to 24 months after unilateral intra-putaminal hRPE-GMs grafts¹⁵. Whereas in another large-scale, multicenter and randomized controlled Phase II clinical trial (in 71 patients with advanced PD) no clinical benefits of this therapy were observed. Compared to the sham group (n=36), the mean motor scores did not differ significantly in hRPE-GM implanted patients (n=35)¹⁴. These discrepant results raise a number of critical issues, which need to be addressed before further clinical trials are performed. Among them, the fact that “no assay is available to detect the activity of hRPE cells following their implantation into the host brain in vivo” is a major deficiency^{13,17}. Neither behavioral evaluation scoring systems nor in vitro assays can provide solid experimental evidence to fully assess hRPE's anti-PD mechanisms of action and to measure the degree of grafted cell survival and activity in the recipient brain directly. Therefore, in order to understand the anti-PD treatment efficacy of hRPE cell implantation and help to resolve the paradox between the positive preclinical findings and the disappointing results of the 1^{st} clinical phase II trial, it is crucial to develop a non-invasive imaging technique to detect accurately the survival and activity of hRPE cells following their implantation into the host brain in vivo. Although many efforts have been undertaken in this area, such an imaging technique has not been successfully developed and remains an unmet medical need.

Nuclear neurotransmitter imaging has been considered a promising tool to monitor intrastrial implanted hRPE cells. Positron emission tomography (PET) combined with molecular probes has been widely used for imaging of the physiological processes of neurotransmitters in the presynaptic, synaptic, and postsynaptic regions of dopaminergic neurons. Up to now, several specific PET probes targeting signaling pathways or biomarkers related to biochemical process of dopamine synthesis and activities have been evaluated for imaging hRPE cells grafted into the striatum. They include ¹¹C-raclopride, which indicates dopamine release, and ¹⁸F-fluoro-L-DOPA, which measures endogenous enzymatic activities related to dopamine processing and storage¹⁸⁻²⁰. However, the efficacy of these probes for hRPE cell tracking is controversial and problematic. Both the changes of dopamine release and enzyme expression are not exclusive to implanted hRPE cells, but could also be attributed to the function of endogenous dopaminergic neurons. The lack of high targeting specificity of these probes results in high imaging background and poor imaging quality. Thus it is very complex to interpret the PET images obtained. These results highlight the urgent need of developing new PET probes that can monitor RPE cells and image their exclusive activities.

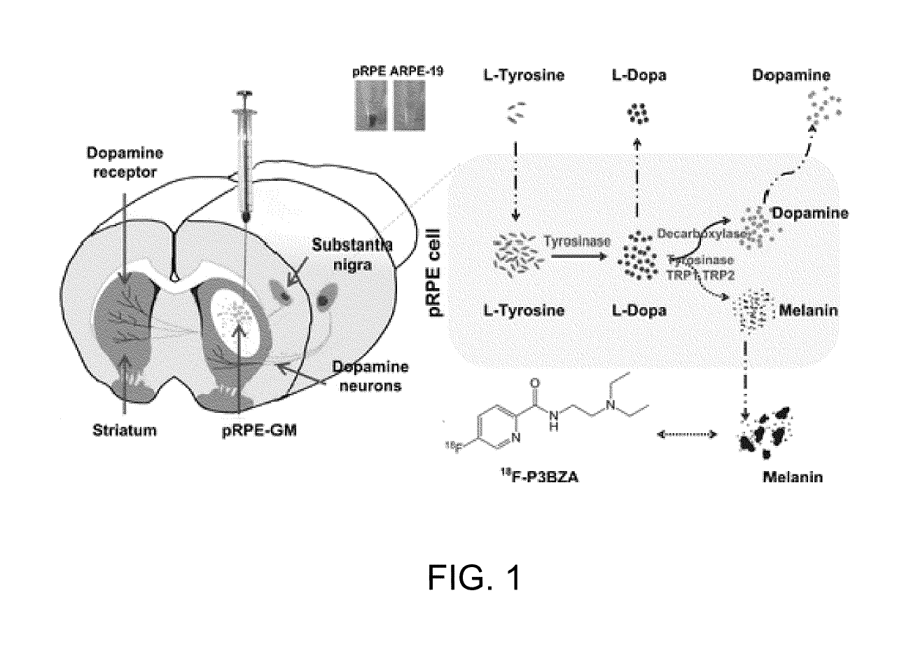

It is well known that hRPE cells feature high melanin content. L-DOPA is a precursor directly involved in the melanin synthesis pathway in cells (FIG. 1)^{21,22}. Considering the main mechanism of RPE cell treatment of PD is the delivery of the melanin synthesis precursor L-DOPA, melanogenesis in RPE cells could thus serve as a surrogate biomarker for their therapeutic activity, and melanin may provide a promising target for in vivo molecular imaging of RPE cells. Recently, we successfully developed a melanin targeted small lipophilic PET probe, N-(2-(diethylamino)ethyl)-¹⁸F-5-fluoropicolinamide (¹⁸F-P3BZA). It can be easily prepared by a one-step radiosynthesis, displays high melanin targeting specificity and shows excellent performance for in vivo imaging of melanotic melanoma^{23,24}.

In the current study, we hypothesized that ¹⁸F-P3BZA could also serve as a valid PET probe for monitoring and tracking RPE cells after intrastriatal transplantation. We longitudinally imaged intrastriatal transplanted RPE cells in rats using PET/CT (computed tomography) (FIG. 1). Specifically, melanotic porcine retinal pigment epithelial (pRPE) cells²⁵ were used as the source of donor cells and pRPE-GMs were prepared and intrastriatally implanted in normal rats. A spontaneously arising RPE cell line²⁶, ARPE-19, which does not normally produce melanin, was also attached to GMs (ARPE-10-GMs) and used as a control. The aims of this study were: (1) to validate the efficacy of ¹⁸F-P3BZA for visualizing the activity of RPE cells by monitoring the metabolic activity of the melanin synthesis pathway in vivo; and (2) to monitor the long-term graft activity of implanted RPE cells following their implantation into the host striatum in vivo via melanin targeting molecular imaging.

### Results

### 1. Synthesis of ¹⁸F-P3BZA

The chemical structure of ⁸F-P3BZA is shown in FIG. 1. High-Performance Liquid Chromatography (HPLC) purification of the radioactive ¹⁸F-P3BZA yielded 9.5±1.9% radiochemical yields with 10.4 minute retention time, affording a product with >95% radiochemical purity (RCP) and specific activity (SA) of 100-150 GBq/μmol.

### 2. In Vitro Cell Uptake Study and Measurement of Melanin

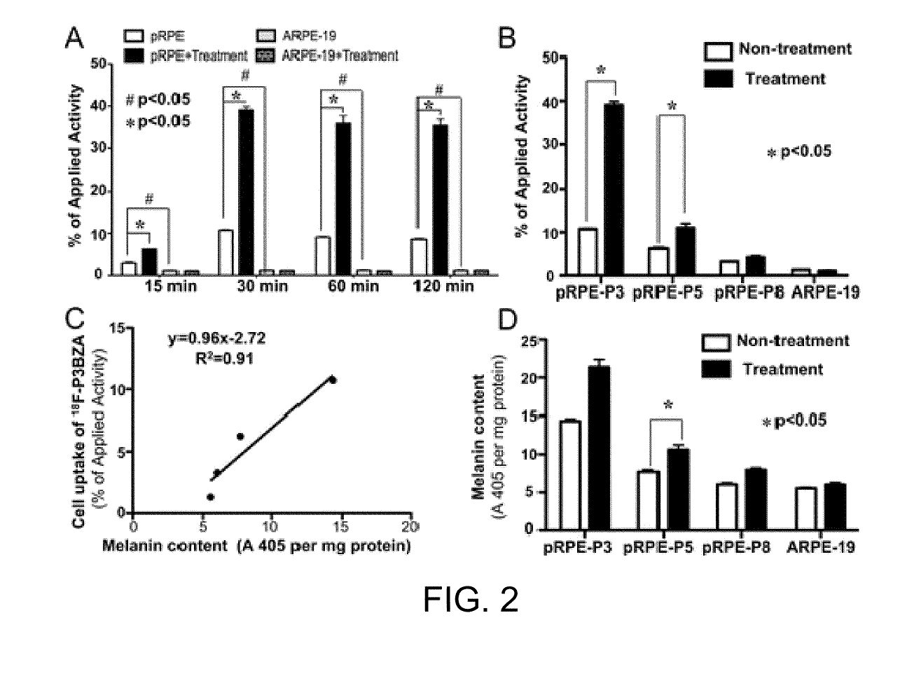

Cell uptake results at multiple time points showed that ¹⁸F-P3BZA accumulated effectively in pRPE cells but not in control ARPE-19 cells (FIG. 2A). The uptake of ¹⁸F-P3BZA in pRPE cells maximized at a mean±standard deviation of 10.72%±0.38 of applied activity within 30 minutes and remained at 9.09%±0.31 and 8.61%±0.47 at 60 and 120 minutes, respectively. At each time point, uptake in pRPE cells was significantly higher than that in the control ARPE-19 cells, which remained low (P=0.03). For pRPE cells, there was a pronounced increase in uptake for ¹⁸F-P3BZA after treatment with tyrosine, which was approximately three- to fourfold higher than that in nontreatment groups. For the control ARPE-19 cells, however, there was no difference before and after tyrosine treatment (P=0.25).

The cell uptake of ¹⁸F-P3BZA in pRPE cells at different passages showed that pRPE-P3 had the greatest uptake, followed by pRPE-P5 and then pRPE-P8 (the decreasing rate is −1.45% of applied activity per passage, P<0.0001), with a decreasing response to tyrosine treatment. After tyrosine treatment, uptake of ¹⁸F-P3BZA in pRPE-P3, pRPE-P5, and pRPE-P8 was increased by 48.8%, 37.8%, and 31.9%, respectively (FIG. 2B). There was no difference for the probe uptake between 1-tyrosine-treated ARPE-19 cells and untreated ARPE-19 cells (P=0.13).

The amount of ¹⁸F-P3BZA uptake (mean, 10.71%±0.38, 6.25%±0.59, 3.29%±0.15, and 1.31%±0.15 of applied activity) was highly correlated with the melanin content (14.34 A405 per milligram of protein±0.48, 7.71 A405 per milligram of protein±0.50, 6.04 A405 per milligram of protein±0.38, and 5.56 A405 per milligram of protein±0.21) in pRPE cells and control ARPE-19 cells (FIG. 2, C, R2=0.91). Melanogenesis was reduced with an increasing number of pRPE cell passages (rate of decrease, −1.57 A405 per milligram of protein per passage; P=0.0011) (FIG. 2D).

### 3. Dynamic PET Scans in Normal Rat Brain

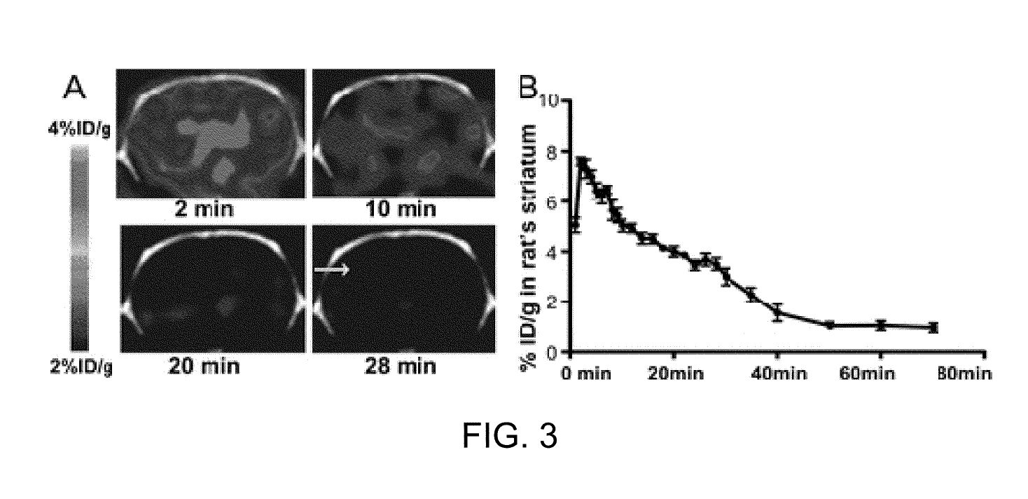

To assess the in vivo brain uptake and clearance of ¹⁸F-P3BZA in normal rat (n=4), dynamic small animal PET scans were initiated immediately after administration of ¹⁸F-P3BZA and terminated 70 minute later. PET images provided visual evidence that ¹⁸F-P3BZA could rapidly cross the blood-brain barrier (BBB) and accumulate in the brain. Moreover, the majority of the probe could be cleared mostly at 28 minute after injection (indicated by yellow arrow, FIG. 3A). Similarly, a time-activity curve (TAC) of brain from dynamic PET demonstrated that ¹⁸F-P3BZA accumulated in the rat brain rapidly (FIG. 3B), peaked at 7.61±0.30 percentage of the injected radioactive dose per gram of tissue (% ID/g) within the first 2 minutes, and then the probe uptake gradually decreased over the remaining time of the scan. The amount of ¹⁸F-P3BZA accumulated in brain decreased to 3.54±0.21% ID/g at 28 minute, and further decreased to 1.04±0.37% ID/g at 60 minute after injection. These results suggest that ¹⁸F-P3BZA has favorable pharmacokinetics, which makes it possible for melanin targeted imaging in the brain.

### 4. In Vivo Small-Animal Static PET/CT Scans, Ex Vivo Autoradiography Study and In Vitro H&E Staining

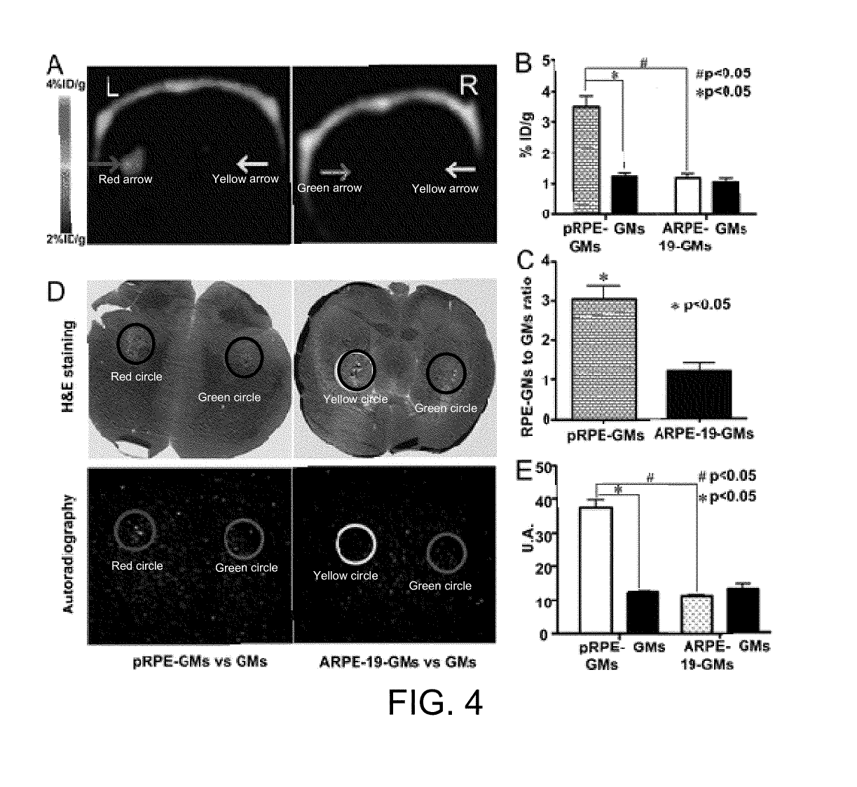

On PET/CT scans, the injected microcarrier-bound pRPE cells were clearly visualized with high contrast, whereas both control gelatin microcarrier-bound ARPE-19 cells and the contralateral control gelatin microcarriers could not be delineated (FIG. 4A). Results of quantification analysis are summarized in FIG. 4B. The uptake of 18F-P3BZA in gelatin microcarrier-bound pRPE cells (mean, 3.48% ID/g±0.58) was higher than that in control microcarrier-bound ARPE-19 cells or gelatin microcarriers alone (1.23% ID/g±0.19 and 1.19% ID/g±0.23, respectively; P=0.003). The gelatin microcarrier-bound pRPE cell-to-control gelatin microcarriers uptake ratio was significantly higher than the gelatin microcarrier-bound ARPE-19 cell-to-gelatin microcarriers uptake ratio (mean, 3.05±0.57 vs 1.21±0.36, respectively; P=0.04) (FIG. 4C).

The implantation sites and the anatomy of brain sections were easily recognized (FIG. 4D). All of the cells and gelatin microcarriers injected were within the striatum and formed clumps.

Autoradiography images also revealed prominent ¹⁸F-P3BZA accumulation in the area of the gelatin microcarrier-bound pRPE cell transplant site. A “hot spot” was evident in the region that exhibited both microcarrier aggregation and structural changes due to surgery in the parallel macroscopic hematoxylin-eosin-stained section. No visible hot spot could be identified in the injection sites for control gelatin microcarrier-bound RPE-19 cells or contralateral gelatin microcarriers alone. Results of quantitative analysis showed that the signal of the transplant site for the gelatin microcarrier-bound pRPE cells was significantly higher than that of injection sites for gelatin microcarrier-bound ARPE-19 cells or contralateral gelatin microcarriers alone (P<0.0001) (FIG. 4E).

### 5. Longitudinal PET/CT Scans

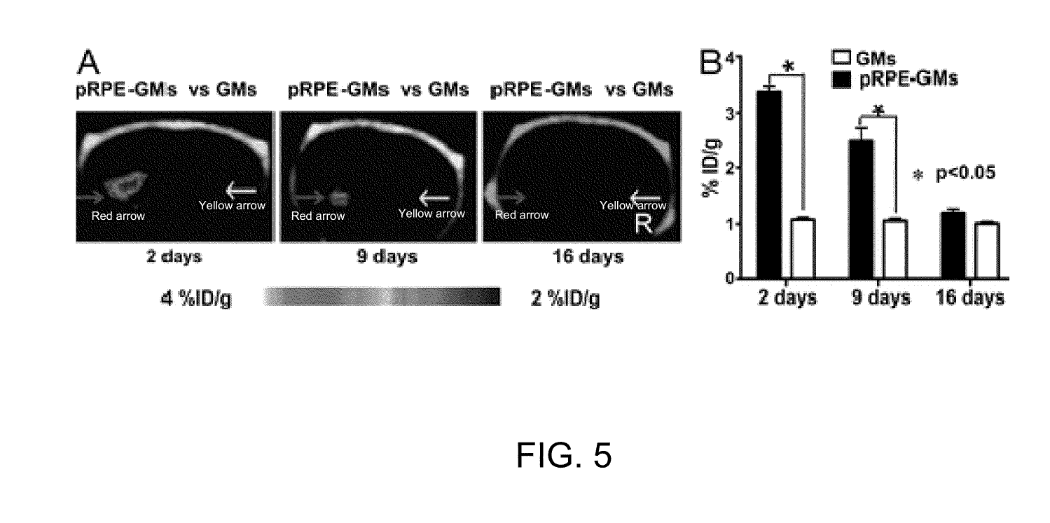

In order to study the long-term survival and activity of implanted RPE cells in vivo, longitudinal PET/CT scans in the same batch of rats were acquired at 2, 9 and 16 days post-implantation of pRPE-GMs in the left striatum and GMs as controls in the right striatum in healthy rats. PET/CT scans were started at 1 h after tail vein injection of ¹⁸F-P3BZA. Representative images are shown in FIG. 5A and the results of quantification analysis of ROIs (transplantation sites) are summarized in FIG. 5B. Clearly, PET/CT imaging demonstrated that ¹⁸F-P3BZA accumulations in pRPE-GMs decreased gradually with time after implantation. The pRPE-GMs were obviously visible as a strong ‘hot spot’ at 2 days post-implantation, whereas the imaging signal of pRPE-GMs was much weaker and showed as a moderate ‘hot spot’ at 9 days post-implantation. At 16 days, cells could not be clearly recognized. Uptake of ¹⁸F-P3BZA peaked at 3.39±0.18% ID/g at 2 days post-implantation, then decreased to 2.49±0.41% ID/g at 9 days, and continued to drop to a baseline level (1.20±0.13% ID/g) at 16 days.

### 6. Fontana-Masson Staining and Immunochemical Staining

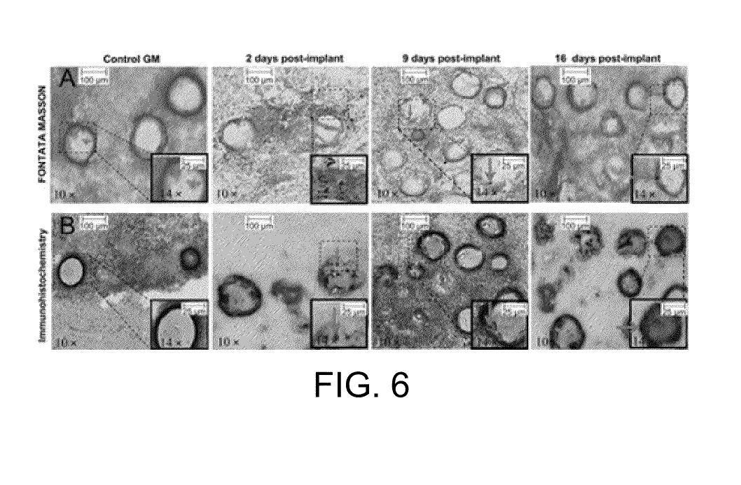

To evaluate histological changes, immunohistochemical staining (IHC) using an RPE-specific marker (RPE65) and Fontana-Masson staining were obtained after longitudinal PET/CT scans at various times post-implantation (FIG. 6). RPE65-positive cells were found to surround the GMs closely at days 2, 9 and 16. Melanin was identified as black spots by Fontana-Masson staining. At day 2 post-implantation, lots of granular melanin spots were seen located both inside and outside of pRPE cells. At day 9, a moderate amount of granular melanin spots were found located in the pRPE cells and a lower level of clustered melanin located outside of the cells. At day 16, a very low number of melanin spots could be found in a small fraction of pRPE cells, and there were several clusters of melanin spots located outside of pRPE cells.

### Discussion

Because there is an urgent need to develop novel imaging techniques to monitor the survival and activity of implanted RPE cells in vivo, we evaluated a melanin targeted PET probe ¹⁸F-P3BZA in a rat model implanted with melanotic pRPE. RPE cells can produce L-DOPA and synthesize melanin and dopamine, making it is feasible to track the implanted RPE cells for anti-PD treatment indirectly by visualizing the melanin with ¹⁸F-P3BZA PET in vivo.

As expected, ¹⁸F-P3BZA shows high specificity localization in pRPE cells effectively in a melanin-depended manner in vitro and in vivo. In vitro, uptake of ¹⁸F-P3BZA in passage 3 pRPE cells was found to be 9-fold higher than that in control amelanotic ARPE-19 cells. L-tyrosine is one of the key substrates of the melanogenic pathway, and typically used to boost melanin content in targeted cells and verify the specificity of melanin-targeting radioactive probes^{27,28}. With 2 mM L-tyrosine pre-treatment, the melanin content in pRPE cells (P3) is significantly increased from 14.34±0.48 to 21.34±1.73 (A 405 per mg protein) (P<0.05). Correspondingly, in vitro cell uptake of ¹⁸F-P3BZA in treated pRPE cells at 30 minute incubation time is significantly elevated comparing to that of the non-treated group (39.07±1.65% vs. 10.72±0.38%, P<0.0001), validating that the ¹⁸F-P3BZA uptake is associated with melanin content in pRPE cells (FIGS. 2A and 2D). Further correlation analysis also demonstrates strong correlation between the uptake of ¹⁸F-P3BZA and melanin content of pRPE cells (R²=0.91, FIG. 2C). In vivo, PET imaging study with rats implanted with cells showed that ¹⁸F-P3BZA could clearly delineate the implanted pRPE cells from the background brain signal at 2 days after implantation (FIG. 4A). Moreover, the accumulation of the PET probe in the pRPE-GMs is much higher than that of ARPE19-GMs or GMs only (P<0.05) (FIGS. 4A, B and C). The autoradiography images combining with H&E staining results further confirm the PET/CT imaging findings, showing that ¹⁸F-P3BZA mainly accumulated in the pRPE cells implanted sites (FIG. 4D). Our results demonstrate that ¹⁸F-P3BZA is an effective probe for imaging RPE cells transplanted into the striatum.

Our study reveals that ¹⁸F-P3BZA PET exhibit excellent imaging quality (FIG. 4A). High pRPE-GMs uptake to contralateral GMs or adjacent normal striatum uptake ratios can be obtained as 3.05±0.57 and 3.19±0.53, respectively. These high uptake ratios can be mainly attributed to the high melanin targeting specificity of ¹⁸F-P3BZA, as well as a lack of endogenous melanin in the rat striatum area. It has been reported that neuromelanin (NM, one form of melanin which is present in brain) is almost absent from the brain in many lower species including rats, but NM appears in high quantities in three main regions of human brain: the substantia nigra of the midbrain, the locus coeruleus within the pons, and the ventrolateral reticular formation and the nucleus of the solitary tract in the medulla oblongata²⁹. Considering the RPE cells are only implanted into the striatum of patients in clinical trials and there is no melanin expression in human striatum, a high RPE cell uptake of ¹⁸F-P3BZA to striatum ratio is expected to be achieved in human, and the presence of melanin in the other three regions will have minimum interference of ¹⁸F-P3BZA PET imaging. The ¹⁸F-P3BZA signals in striatum thus can be used to measure the activity of implanted RPE cells. This is a major advantage of ¹⁸F-P3BZA over other PET probes such as the previously reported ¹¹C-raclopride or ¹⁸F-fluoro-L-DOPA PET¹⁸⁻²⁰. For these two probes, the endogenous dopamine or enzymatic activities could compromise their performance for imaging of implanted RPE cells. The imaging quality of these two probes is quite poor. Before and after implantation of the RPE cells, the implantation site (striatum) to cortex uptake ratios are only 1.35±0.10 and 1.48±0.07 for ¹⁸F-fluoro-L-DOPA, and 2.41±0.74 and 2.44±0.67 for ¹¹C-racloprideat, respectively¹⁹. More importantly, ¹¹C-raclopride and ¹⁸F-fluoro-1-DOPA PET imaging represent the overall information from endogenous dopamine release or enzymatic activities and the changes caused by implanted RPE cells. Thus, it is very hard to use them for evaluating the treatment efficacy of RPE cells.

In the present study, longitudinal PET/CT scans also reveal that the activity of implanted pRPE cells decreases following their implantation into the host, as evident by the significant reduced uptake of ¹⁸F-P3BZA in the pRPE-GMs (FIG. 5). The gradually reduced ¹⁸F-P3BZA uptakes in pRPE-GMs over time (day 2, 9, and 16) suggest that the total melanogenesis in implanted cells is decreasing. Such a consequence likely results from either cell death after implantation or diminished melanogensis ability/activity of the surviving cells, or both. To further pin down the reasons, IHC and Fontana-Masson staining were performed to identify pRPE cells and melanin in the implanted rat brains (FIG. 6). Interestingly, the IHC results are inconsistent with those from Fontana-Masson staining. The distribution pattern and amount between pRPE cells and melanin do not match with each other. The IHC staining using antibodies against RPE65 demonstrates that an appreciable number of implanted pRPE cells survive over the 16-days experimental period, with the number of cells only slightly reduced. A similar cell survival pattern has been reported in studies using other types of RPE cell donors and animal models^{11,12,30}. However, Fontana-Masson staining results revealed that the amount of melanin decreases sharply over time. At day 16, melanin cannot be found in a majority of pRPE cells, other than small clusters of melanin located outside pRPE cells. Because many pRPE cells still survive at 16 days, it is likely that the diminished uptake of ¹⁸F-P3BZA is caused by the reduced activity of melanin synthesis in pRPE cells as cells divide. Our investigation of in vitro ¹⁸F-P3BZA uptake and melanin expression in pRPE cells at different passages (P3, P5 and P8) provide evidence to support this conclusion. Indeed as pRPE cell passages advance, melanin content decreases, and the uptake of ¹⁸F-P3BZA reduces accordingly (FIGS. 2B and D). The ¹⁸F-P3BZA uptake shows an excellent correlation with melanin content in pRPE cells at different passages (FIG. 2C, R²=0.91).

A survey of the available literature shows that melanogenesis in adult RPE cells remains a matter of controversy. Some scientists conclude that melanin synthesis occurs in hRPE cells throughout a person's life albeit at a slow rate, while others consider that it ceases right at birth of a person^{8,31}. More specifically, age-related changes of melanin in hRPE cells was studied and reported³². The authors analyzed hRPE cells from donors of different ages and found an age-related melanin loss in hRPE cells, with melanin content diminishing 2.5-fold between children (<10 year) and old people (>90 year). In another study, the authors demonstrated that dopaminergic communication only takes place in the embryonic retina, indicating that RPE is a potential source of L-DOPA in the early development¹⁰. In our current study, adult pRPE cells have been adopted as source of donors, and we have found that melanin production decreases over advancing passages. Because the activity of the melanin synthesis pathway may be a surrogate biomarker of L-DOPA synthesis, which provides a rationale for the application of hRPE cells in anti-PD therapy, the source of hRPE cell donors is critical for successful therapy. Most previous reports of hRPE cell therapy for PD do not define standard criteria for selection of donors. Using cells obtained from donors of different ages may be partly responsible for the variation and inconsistencies in the treatment outcomes from different preclinical and clinical studies.

Some limitations or concerns should be noted for the imaging technique developed in our study. First, some scientists did report the poor overall survival of implanted cells³⁰, which could partly contribute to the lack of postoperative benefit in some subjects. ¹⁸F-P3BZA PET is a powerful tool to monitor the efficacy of RPE cell transplantation therapy by detecting total melanogenesis, while it clearly cannot differentiate the two processes from each other that determine the overall melanin expression level: melanin loss caused by cell death and poor melaninogenesis activity. But this limitation is likely not to cause a problem since both poor cell survival and low melaninogenesis ability reduce the treatment efficacy of cells. Other imaging techniques that could monitor the cell survival may be developed to further improve anti-PD therapy using RPE cells. A second limitation is that we did not obtain postmortem evidence about the correlation between melaninogenesis and L-DOPA or dopamine production by implanted cells in the treated subjects. This is mainly because of the difficulty to differentiate and quantify exogenous L-DOPA or dopamine from endogenous molecules in vivo. But we did provide evidence that pRPE can produce melanin in vitro and in vivo (FIG. 2D and FIG. 6). It has been found that in vitro cultured RPE cells do express L-DOPA decarboxylase and can synthesize dopamine^{21,33}. A third limitation is that our study is based on a rat model with porcine RPE cells as the source of donor cells. It is necessary to further study the efficacy of ¹⁸F-P3BZA PET on humans or non-human primates using allografts hRPE cells. Regardless of the above limitations, our study for the first time validated ¹⁸F-P3BZA as a melanin-dependent PET probe with high potential for monitoring the activities of implanted pRPE cells. The promising imaging results elucidated in the present study suggest the usefulness of ¹⁸F-P3BZA PET for molecular imaging of RPE cells following their implantation into a host brain. This is the first study as well to assess the activity of the melanin synthesis pathway in pRPE cells in vivo by the non-invasive PET imaging technique.

In conclusion, ¹⁸F-P3BZA is able to identify and bind to porcine RPE cells specifically and effectively through melanin targeting in vitro and in vivo. ¹⁸F-P3BZA PET/CT provides unprecedented opportunities to visualize, characterize and detect the long-term activity of implanted RPE cells. It may contribute to improving the therapeutic efficacy of hRPE cells for PD treatment in the clinic.

### Materials and Methods

### General

All chemicals were purchased from Sigma-Aldrich (St. Louis, Mo.) unless otherwise stated. No-carrier-added ¹⁸F-fluoride was produced by the ¹⁸O (p, n)¹⁸F nuclear reaction on a PETtrace cyclotron (GE Medical Systems; Milwaukee, Wis.). ¹⁸F-P3BZA was prepared on a GE TRACERLab FX FN module (GE Medical Systems; Milwaukee, Wis.). Pig eyeballs were purchased from Animal Technologies Inc. for isolation of pRPE cells. The amelanotic hRPE cell line, ARPE-19, was used as a control and obtained from American Type Culture Collection (ATCC, Manassas, Va., USA).

### Radiosynthesis of ¹⁸F-P3BZA

Melanin targeted PET probe ¹⁸F-P3BZA, was prepared according to our previously published procedure^{23,24}. Briefly, 5-bromo-N-(2-(diethylamino)ethyl)-picolinamide (5 mg) was reached with K¹⁸F-K_{2.2.2} complex in 200 μL anhydrous DMSO at 110° C. for 10 minute with stirring. The radiolabeled product was isolated by a semi-preparative HPLC, and the fractions containing the product were collected and combined. The final product ¹⁸F-P3BZA was reconstituted in phosphate buffered saline (PBS, 0.1 M, pH=7.4). To determine specific activity, radiochemical yields and purity, the final product was injected into an analytical HPLC with the flow rate of 1 mL/minute using a Vydac protein and peptide column (218TP510; 5 μm, 250×4.6 mm) The UV absorbance was monitored at 218 nm and the identification of the small molecules was confirmed based on the UV spectrum acquired using a PDA detector. Before use in vitro and in vivo animal experiments, the final radioactive product was passed through a 0.22 μm filter into a sterile vial.

pRPE Cells Isolation and Culturing

Melanotic pRPE and amelanotic ARPE-19 cells were used as positive and negative controls, respectively, for evaluation of ¹⁸F-P3BZA in vitro and in vivo. pRPE cells were isolated in our lab according to the procedure previously published³⁴. Briefly, fresh eyeballs were cleaned by removing all surrounding tissues. The eyeballs were sterilized by incubating in betadine solution (Purdue Products, Stamford, Conn.) for 15 minute and then washed with sterilized PBS. The anterior part of the eye and the neural retina, approximately 3.5 mm posterior to the limbus was removed, following by adding 0.25% trypsin to the posterior eyecup and incubating at 37° C. for 1 h. The enzyme solution was pipetted up and down a few times to loose pRPE cells from choroid/sclera, and the pRPE cells were collected into a centrifuge tube. Culture medium [Dulbecco's modified Eagle high-glucose medium with low glucose (DMEM-low-glucose), 10% FBS, antibiotic/antimycotic) (Gibco Life Sciences)] was added to the cells to stop enzyme reaction. To obtain pure pRPE population, the cells suspension was placed on the top of 40% Percoll cushion (in PBS) and centrifuged for 10 minute at 300 g. Because pRPE cells are pigmented, they were sediment to the bottom of the centrifuge tube. The purified pRPE cells were resuspended into the culture medium and plated in culture dishes. Cell cultures were incubated at 37° C. with 5% CO₂ and medium was changed 2-3 times a week. Both pRPE and hRPE cells were cultured using the same culture medium and incubator, and grown to 100% confluence before GM attachment and implantation.

### In Vitro Assays

### Cell Uptake of ¹⁸F-P3BZA

pRPE (generation 3, 5×10⁵) or control ARPE-19 cells were plated in a 12-well plate pretreated with or without L-tyrosine (2 mM) for 24 h. They were incubated with 74 kBq (2 μCi)/well of ¹⁸F-P3BZA at 37° C. for 15, 30, 60, and 120 minute. The cells were washed three times with chilled PBS and lysed with 500 μL of 0.1 M NaOH. The cell lysates were then collected and the radioactivity of the lysates was measured with a γ counter (Perkin-Elmer, MA, USA). The cell uptake was expressed as the percentage of total applied radioactivity. All experiments were performed with triplicate samples. Similarly, uptake of ¹⁸F-P3BZA at 30 minute incubation was determined in pRPE cells at different passages (pRPE-P3, pRPE-P5 and pRPE-P8) and control ARPE19 cells, with or without L-tyrosine pretreatment.

### Melanin Quantification Analysis

In vitro measurement of melanin content was performed for pRPE and ARPE-19 cells in parallel with the ¹⁸F-P3BZA cell uptake assay according to methods reported previously with slight modifications³⁵. The cells were harvested after pretreatment with or without 2 mM L-tyrosine for 24 h and washed with PBS, then they were incubated overnight in 500 μL of 1 N NaOH at room temperature. The solution was pipetted repeatedly to homogenize the extracts. The concentration of total protein was determined using a microBCA protein assay kit (Pierce Biotechnology) and then the concentration of samples was adjusted to 0.4 μg/μL. Extracts (100 μL) were added into 96-well plates in triplicate. The relative melanin content of samples was determined by measuring their absorbance at 405 nm using a plate reader (TECAN, Research Triangle Park, N.C.). Results were expressed as absorbance of 405 nm per mg protein (A 405 nm/mg protein).

### Cell Implantation

### Attachment of Cells to GMs

RPE cells (pRPE and control ARPE-19) were passively absorbed or attached to GMs (Cytodex® 1, 131-220 μm, dextran beads, Sigma) according to the typical methods¹¹. Briefly, 24 h before the attachment study, dry GMs were hydrated in a 1.5-mL microcentrifuge tube with calcium/magnesium-free PBS (Invitrogen) for a minimum of 1.5 h and then autoclaved (121° C., 15 psi, 30 minute) for sterilization. Sterilized GMs were then re-suspended and washed twice with fresh PBS and stored in complete pRPE medium. After harvested by trypsinization and mechanical agitation, RPE cells (≧90% cell viability) was mixed with GMs in complete RPE medium to obtain the suspension with a final concentration of 1×10⁶ cells/10 mg of GMs in a 1.5 mL micro-centrifuge tube, and the resulting mixture was placed for 15-18 hours in 37° C. incubator. After incubation, the RPE-GMs suspensions were gently washed with PBS to remove any unattached RPE cells. A small volume of (10 μL) sample was taken and treated with a Dispase solution (Sigma Aldrich, St Louis, Mo., USA) to break down GMs, and the samples were assessed for cell viability and concentration using the trypan blue exclusion method. Only RPE-GM suspensions passing the minimum criteria (≧90% cell viability and ≧2000 cells/μL RPE-GM suspension) were selected for implantation. GM suspensions were treated in a similar manner as controls.

### 5.2 Surgical Procedure

Female Wistar rats (Charles Rivers Laboratory, Wilmington, Mass., USA) weighing 176-200 g were used in this study. All animal work was conducted in accordance with the Administrative Panel on Laboratory Animal Care at Stanford University. The implantation procedure was performed following the surgical procedures described in a previous report³⁶. Animals were anesthetized using isofluorane and were implanted at left striatum with RPE-GMs using the following (flat-skull) coordinates: anterior posterior (AP) −0.4; medial lateral (ML) ±3.5; dorsal ventral (DV) −5.0. The control GMs-alone were implanted on the contralateral side of the striatum and performed using the same procedure. Before the implantation procedure, solutions contained RPE cells (melanotic pRPE or amelanotic ARPE-19, 12,000˜15,000 cells in 10 μL) were drawn into a sterile 50 μL Hamilton syringe (Hamilton Company, Reno, Nev.).

### 6. PET/CT Imaging

Small animal PET/CT imaging was performed on a Siemens Inveon small-animal multimodality PET/CT system (Preclinical Solutions; Siemens Healthcare Molecular Imaging, Knoxville, Tenn.). For each rat, ¹⁸F-P3BZA (7.4 MBq, 200 μCi) was administrated through tail vein.

### Dynamic PET Imaging

To assess the in vivo kinetics of ¹⁸F-P3BZA in normal rat brain, dynamic PET imaging (10×1 minute, 10×2 minute, 2×5 minute, 3×10 minute; total of 25 frames) was initiated immediately after administration of the probe and terminated 70 minute later on normal rats (n=4).

CT scan (632 slices at 206 μm) was performed immediately after PET imaging for both photon attenuation correction and image co-registration with PET image data for anatomical information. PET Images were reconstructed with a 2-dimensional ordered-subsets expectation maximization algorithm (OSEM 2D). ROIs were drawn in bilateral striatum using the Inveon Research Workspace software. The average radioactivity concentration in the ROIs was obtained from the mean pixel values within the ROIs volume. These data was converted to counts per milliliter per minute by using a predetermined conversion factor. The results were then divided by the injected dose to obtain an image ROI-derived % ID/g, and the TAC was then calculated and obtained.

### Static PET/CT Imaging

To investigate the efficacy of ¹⁸F-P3BZA for imaging of implanted pRPE cells in vivo, static PET scans (10-minute) was performed on normal rats with pRPE-GMs (n=4) or ARPE-19-GMs (n=4) implanted in left striatum, parallel with GMs only as controls in the right striatum. PET/CT scans were performed at 1 h after tail vein injection of ¹⁸F-P3BZA. Rats were sacrificed immediately after the PET/CT scan, and rat brain sections were prepared for autoradiography and HE staining

### Longitudinal PET/CT Imaging Studies

To evaluate the potential of ¹⁸F-P3BZA for monitoring the survival and function of RPE cells implanted, longitudinal PET/CT scans were acquired at day 2, 9 and 16 after cell implantation of pRPE-GMs in the left and GMs as control in right striatum in normal rats (n=12). PET/CT imaging started at 1 h after tail vein injection of ¹⁸F-P3BZA Immediately after PET/CT scan at each time point (day 2, 9 and 16), four rats were sacrificed and the brain slices were prepared for immunohistochemistry and Fontana-Masson staining.

### Postmortem Analysis

The sacrificed rat brains were removed and embedded in optimum cutting temperature (O.C.T.) compound (Tissue-Tek, Sakura, USA). Subsequently, thick axial blocks (12 μm) were cut using a cryostat microtome HM500 (Microm, HM 500 M, Heidelberg, Germany). The sections were mounted on microscope slides (Fisherbrand Superfrost Plus microscope slides, Fisherbrand Superfrost Plus Microscope Slides, Vernon Hills, Ill., USA). The representative coronal sections through the striatum (including visible injection sites) were used for autoradiography immediately or preserved in −80° C. for other postmortem analysis.

### Ex Vivo Autoradiography

After static PET/CT imaging, frozen brain sections were prepared immediately for autoradiography. The sections were air-dried for 10 minute, and then exposed to a radioactivity sensitive storage phosphor screen (Perkin-Elmer, USA) for 24 h at 4° C. The imaging plates were analyzed using a Typhoon 9410 variable mode imager (Amersham Biosciences, Salt Lake City, Utah, USA), and the image data were visualized and processed by Image J (image processing and analysis software in Java).

### H&E Staining

After static PET/CT imaging, the brain sections adjacent to those for autoradiography study, were stained with hematoxylin and eosin using H&E Stain Kit (BBC Chemical, TX, USA) and following the instruction to identify the implantation sites and the anatomy of brain sections.

### Fontana-Masson Staining

After longitudinal PET/CT imaging, sections from the block containing a representative RPE cell profile on initial review were stained with Fontana-Masson staining (AMTS Inc., Lodi, Calif., USA). Fontana-Masson staining (FMS) was conducted at each time point to verify the presence of melanin. The staining was performed according to the manufacturer's recommendations step by step with slight modifications. Fontana silver solution was prepared by adding the concentrated ammonium hydroxide solution drop by drop to a mixture with one vial of Fontana silver solution and 27 mL distilled water (10% silver nitrate solution), accompanied by continuous stirring. Frozen tissue slides were fixed in pre-cooled acetone (−20° C.) for 10 minute, then the fixative were poured off and acetone was allowed to evaporate from the tissue sections for >20 minute at room temperature. The tissue sections were quickly rinsed with running tap water followed by another thoroughly rinse with distilled water. The slides were placed in ammoniacal silver solution and incubated in a 58-60° C. water bath for 35 minute. After incubation, the slides were gently rinsed with distilled water and were placed in 0.1% gold chloride for 1 minute. The tissue sections were then rinsed twice with distilled water and placed in 5% sodium thiosulfate for 1-2 minute. After gently rinsed twice in running tap water for 2-3 minute, the slides were placed in Nuclear Fast Red Stain (Sigma Aldrich, St Louis, Mo., USA) for 5 minute, followed by gently rinses with running tap water and dehydration with 3 changes of fresh absolute alcohol. The slides were cleaned through 3 changes of fresh xylene or xylene substitute and covered with a coverslip using a permanent mounting media.

### Immunohistochemical Staining

To identify the implanted pRPE cells, tissue sections adjacent to those for FMS were underwent IHC staining, using RPE65 (Chemicon, Temecula, Calif., USA), a mouse monoclonal antibody (MAb) that specifically reacts to a 65-kD protein on RPE cell membranes. The staining was performed according to the manufacturer's recommendations. After 10-minute fix in ice-cold acetone (−20° C.) and 20 minute air drying at room temperature, the sections parallel to Fontana-Masson staining were quenched in 3% hydrogen peroxide in PBS for 15 minutes, and they were subsequently treated with a blocking solution containing 0.5% Tween-20 and 10% normal donkey serum. Sections were incubated overnight in a humidified chamber at 4° C. with anti-RPE65 MAb (1:10) diluted in blocking solution. On the next day, sections were washed 3 times with PBS containing 0.05% Tween-20 for 3 minute each and treated with a donkey anti-mouse secondary antibody (1:300; Jackson Immunoresearch, West Grove, Pa., USA) in the presence of 2% goat serum for 1 h at room. The slides were washed 3 times with distilled water and were covered with a coverslip using a permanent mounting media. At last, all the slices (except for the autoradiography study) were scanned with a Histalim-Hamamatsu's Nanozoomer slide scanner (Histalim, Montpellier, France).

### Statistical Methods

Quantitative data were expressed as mean±SD. Means were compared using the Student t test. A 95% confidence level was chosen to determine the significance between groups, with P values of less than 0.05 indicating significant differences. The Pearson correlation coefficients (R²) were calculated to assess the relationship of ¹⁸F-P3BZA cell uptake and melanin content in pRPE cells.

### REFERENCES

1. de Lau L M, Breteler M M. Epidemiology of Parkinson's disease. Lancet neurology 2006; 5:525-35.
2. Olanow C W. Levodopa/dopamine replacement strategies in Parkinson's disease—future directions. Movement disorders: official journal of the Movement Disorder Society 2008; 23 Suppl 3:S613-22.
3. Poewe W, Mahlknecht P, Jankovic J. Emerging therapies for Parkinson's disease. Current opinion in neurology 2012; 25:448-59.
4. Olanow C W, Obeso J A, Stocchi F. Continuous dopamine-receptor treatment of Parkinson's disease: scientific rationale and clinical implications. Lancet neurology 2006; 5:677-87.
5. Hayes M W, Fung V S, Kimber T E, O'Sullivan J D. Current concepts in the management of Parkinson disease. The Medical journal of Australia 2010; 192:144-9.
6. Roffler-Tarlov S, Liu J H, Naumova E N, Bernal-Ayala M M, Mason C A. L-Dopa and the albino riddle: content of L-Dopa in the developing retina of pigmented and albino mice. PloS one 2013; 8:e57184.
7. Tombran-Tink J, Shivaram S M, Chader G J, Johnson L V, Bok D. Expression, secretion, and age-related downregulation of pigment epithelium-derived factor, a serpin with neurotrophic activity. The Journal of neuroscience: the official journal of the Society for Neuroscience 1995; 15:4992-5003.
8. Schraermeyer U, Kopitz J, Peters S, et al. Tyrosinase biosynthesis in adult mammalian retinal pigment epithelial cells. Experimental eye research 2006; 83:315-21.
9. Dorey C K, Torres X, Swart T. Evidence of melanogenesis in porcine retinal pigment epithelial cells in vitro. Experimental eye research 1990; 50:1-10.
10. Kubrusly R C, Guimaraes M Z, Vieira A P, et al. L-DOPA supply to the neuro retina activates dopaminergic communication at the early stages of embryonic development. Journal of neurochemistry 2003; 86:45-54.
11. Flores J, Cepeda I L, Cornfeldt M L, O'Kusky J R, Doudet D J. Characterization and survival of long-term implants of human retinal pigment epithelial cells attached to gelatin microcarriers in a model of Parkinson disease. Journal of neuropathology and experimental neurology 2007; 66:585-96.
12. Subramanian T, Marchionini D, Potter E M, Cornfeldt M L. Striatal xenotransplantation of human retinal pigment epithelial cells attached to microcarriers in hemiparkinsonian rats ameliorates behavioral deficits without provoking a host immune response. Cell transplantation 2002; 11:207-14.
13. Watts R L, Raiser C D, Stover N P, et al. Stereotaxic intrastriatal implantation of human retinal pigment epithelial (hRPE) cells attached to gelatin microcarriers: a potential new cell therapy for Parkinson's disease. Journal of neural transmission Supplementum 2003:215-27.
14. Gross R E, Watts R L, Hauser R A, et al. Intrastriatal transplantation of microcarrier-bound human retinal pigment epithelial cells versus sham surgery in patients with advanced Parkinson's disease: a double-blind, randomised, controlled trial. Lancet neurology 2011; 10:509-19.
15. Stover N P, Bakay R A, Subramanian T, et al. Intrastriatal implantation of human retinal pigment epithelial cells attached to microcarriers in advanced Parkinson disease. Archives of neurology 2005; 62:1833-7.
16. Stover N P, Watts R L. Spheramine for treatment of Parkinson's disease. Neurotherapeutics: the journal of the American Society for Experimental NeuroTherapeutics 2008; 5:252-9.
17. Albanese A. Cell therapy for Parkinson's disease: have the glory days gone? Lancet neurology 2011; 10:492-3.
18. Wang R, Zhang J, Guo Z, et al. In-vivo PET imaging of implanted human retinal pigment epithelium cells in a Parkinson's disease rat model. Nuclear medicine communications 2008; 29:455-61.
19. Doudet D J, Cornfeldt M L, Honey C R, Schweikert A W, Allen R C. PET imaging of implanted human retinal pigment epithelial cells in the MPTP-induced primate model of Parkinson's disease. Experimental neurology 2004; 189:361-8.
20. Yin F, Tian Z M, Liu S, et al. Transplantation of human retinal pigment epithelium cells in the treatment for Parkinson disease. CNS neuroscience & therapeutics 2012; 18:1012-20.
21. McKay B S, Goodman B, Falk T, Sherman S J. Retinal pigment epithelial cell transplantation could provide trophic support in Parkinson's disease: results from an in vitro model system. Experimental neurology 2006; 201:234-43.
22. Zareba M, Raciti M W, Henry M M, Sarna T, Burke J M. Oxidative stress in ARPE-19 cultures: do melanosomes confer cytoprotection? Free radical biology & medicine 2006; 40:87-100.
23. Liu H, Liu S, Miao Z, et al. Development of 18F-labeled picolinamide probes for PET imaging of malignant melanoma. Journal of medicinal chemistry 2013; 56:895-901.
24. Qin C, Cheng K, Chen K, et al. Tyrosinase as a multifunctional reporter gene for Photoacoustic/MRI/PET triple modality molecular imaging. Scientific reports 2013; 3:1490.
25. Zhang H L, Wu J J, Ren H M, Wang J, Su Y R, Jiang Y P. Therapeutic effect of microencapsulated porcine retinal pigmented epithelial cells transplantation on rat model of Parkinson's disease. Neuroscience bulletin 2007; 23:137-44.
26. Dunn K C, Aotaki-Keen A E, Putkey F R, Hjelmeland L M. ARPE-19, a human retinal pigment epithelial cell line with differentiated properties. Experimental eye research 1996; 62:155-69.
27. Kim H J, Kim D Y, Park J H, et al. Synthesis and characterization of a (68)Ga-labeled N-(2-diethylaminoethyl)benzamide derivative as potential PET probe for malignant melanoma. Bioorganic & medicinal chemistry 2012; 20:4915-20.
28. Ren G, Miao Z, Liu H, et al. Melanin-targeted preclinical PET imaging of melanoma metastasis. Journal of nuclear medicine: official publication, Society of Nuclear Medicine 2009; 50:1692-9.
29. Fedorow H, Tribl F, Halliday G, Gerlach M, Riederer P, Double K L. Neuromelanin in human dopamine neurons: comparison with peripheral melanins and relevance to Parkinson's disease. Progress in neurobiology 2005; 75:109-24.
30. Farag E S, Vinters H V, Bronstein J. Pathologic findings in retinal pigment epithelial cell implantation for Parkinson disease. Neurology 2009; 73:1095-102.
31. Schraermeyer U. Does melanin turnover occur in the eyes of adult vertebrates? Pigment cell research/sponsored by the European Society for Pigment Cell Research and the International Pigment Cell Society 1993; 6:193-204.
32. Sarna T, Burke J M, Korytowski W, et al. Loss of melanin from human RPE with aging: possible role of melanin photooxidation. Experimental eye research 2003; 76:89-98.
33. Ming M, Li X, Fan X, et al. Retinal pigment epithelial cells secrete neurotrophic factors and synthesize dopamine: possible contribution to therapeutic effects of RPE cell transplantation in Parkinson's disease. Journal of translational medicine 2009; 7:53.
34. Feng W, Yasumura D, Matthes M T, LaVail M M, Vollrath D. Mertk triggers uptake of photoreceptor outer segments during phagocytosis by cultured retinal pigment epithelial cells. The Journal of biological chemistry 2002; 277:17016-22.
35. Ishikawa M, Kawase I, Ishii F. Glycine inhibits melanogenesis in vitro and causes hypopigmentation in vivo. Biological & pharmaceutical bulletin 2007; 30:2031-6.
36. Cepeda I L, Flores J, Cornfeldt M L, O'Kusky J R, Doudet D J. Human retinal pigment epithelial cell implants ameliorate motor deficits in two rat models of Parkinson disease. Journal of neuropathology and experimental neurology 2007; 66:576-84.

### Example 2

### In Vivo Detection of Neuromelanin and Iron by ¹⁸F-P3BZA PET/MRI—a New Approach for Early Detection of Parkinson Disease

### Introduction

Parkinson's disease (PD) is a common neurodegenerative disorder, which is predominantly driven by the selective and progressive loss of neuromelanin (NM)-containing dopaminergic neurons in the substantia nigra (SN) pars compacta resulting in profound depletion of dopamine in striatal terminal¹. The clinical diagnosis depends on demonstration of symptoms, such as bradykinesia, but no well-established diagnostic biomarker².

Early diagnosis of pre-motor PD, or even at risk, is critical because it would enable effective disease-modifying therapy at early stage, when the greatest number of nerve cells could responsive to intervention³. Many studies showed that PD—specific pathogenic features antedate the onset of diagnostic clinical features⁴. Clinical signs of PD will not appear until profound striatal dopamine deficiency when striatal dopamine is reduced by about 80%⁵. Due to the early symptoms of Parkinson's disease are subtle and occur gradually, so far there are no medical tests to definitively diagnose the disease at the early stage accurately. Therefore, the need for finding diagnostic biomarkers associated with disease specific changes that enable the identification of subjects at early stage are great and acute.

In vivo detection of pathophysiological changes associated with PD is a promising for both the diagnosis of the disease and monitoring response to neuroprotective therapy. Several studies over the past decades have shown that molecular brain imaging targeting to catecholaminergic systems with PET or SPECT allow effective visualization and quantitative measurements of relevant biological and neurochemical processes in PD for diagnosis⁶⁻⁸. Nuclear neurotransmitter imaging is considered as a valuable tool for evaluating the density and affinity of postsynaptic receptors for neurotransmitters such as dopamine, as well as presynaptic transporters for these transmitters, precursors such as L-DOPA and transmitter degrading enzymes⁶. These probes include ¹¹C-raclopride, which indicates dopamine release⁹, ¹⁸F-fluoro-L-DOPA, which measures endogenous enzymatic activities related to dopamine processing and storage¹⁰. Such an approach has provided reliable information about neurochemical abnormalities involved in advanced PD, as well as helping to elucidate the mechanism of action of the pharmacological agents commonly used to treat these conditions. However, the imaging methods based on striatal dopamine deficiency is not sensitive and accurate enough for diagnosis of early stage PD. For instance, ideally, decreased ¹⁸F-fluoro-L-DOPA PET always means the decline of the dopaminergic terminal density and the activity of the aromatic amino acid decarboxylase enzyme (AADC)^{11,12}. The odd thing is that several studies reported increased cortical ¹⁸F-fluoro-L-DOPA uptake at early stage of PD as compared to advanced stage patients and normal controls. The reason may be that the increased ¹⁸F-fluoro-L-DOPA uptake observed in early PD reflects compensatory upregulation as a result of increased activity of AADC in the remaining dopaminergic terminals. Yet, this increase in probe uptake may underestimate the extent of degenerative process at the early PD. On the other hand, the correlation between the loss of striatal probe binding and disease severity is poor. Moreover, unlike in advanced PD, the changes of both dopamine release and enzyme expression are subtle in early stage PD and it is even difficult to track.

It's worth mentioning that there are several nuclear imaging methods beyond the striatonigral dopaminergic system¹³, including imaging the serotonergic system (such as ¹²³I-FP-CIT, which visualize the presynaptic dopamine transporter (DTA) distribution within the striatum)¹⁴, imaging the noradrenergic system (such as ¹¹C-RTI-32, which binding in LC), imaging the cholinergic system (such as ¹²³I-iodobenzovesamicol, which binding in cholinergic nerve terminals and consequently a biomarker of the presynaptic cholinergic terminal density)^{15,16}, imaging Lewy Body (such as ¹¹C-BF227, which binding specificly to Lewy bodies)¹⁷, imaging microglial activation (such as ¹¹C-PK11195, which as an in vivo marker of microglial activation in neurological diseases including PD act as an in vivo marker of microglial activation in neurological diseases including PD)¹⁸ and imaging glucose metabolism (such as ¹⁸F-FDG, which evaluate a network of Parkinson's disease-related pattern of metabolic alterations)^{19,20}. The mechanisms of these imaging methods are based on the fact that PD is a multisystem degenerative process, affecting not only the most targeted nigrostriatal dopaminergic system, responsible for the majority of the motor symptoms, but also other neurotransmitter systems, like cholinergic, serotoninergic, noradrenergic etc. associated with the emergence of a multitude of non-motor symptoms. These methods are no doubt playing a supporting role for PD's diagnosis, but with a negligible shortcoming lack of specificity.

The results above highlight the urgent need of developing novel neuroimaging associated PD pathogenesis for detecting early stage PD and evaluating disease progression.

### Technical Discussion

Neuromelanin (NM) is a dark polymer pigment, which shows the iron chelating capacity and is produced mainly by catecholaminergic neurons in substantia nigra (SN) and the locus coeruleus (LC). Many studies show that the changes of neuromelanin (NM) and iron content are associated with both PD pathogenesis and progression^{1,21}. Seminal studies have demonstrated that the preclinical period of nigral cell loss has been estimated to last several years. By the appearance of the first symptom, there is 60-70% of dopaminergic substantia nigra (SN) neurons have already degenerated. So neuromelanin and iron appears to be a suitable surrogate for neuroimaging of early stage PD.

There is a hypothesized link between the change of NM content and the severity of the specific loss of catecholaminergic neurons in SN²². Until now, there exists no way to detect the content of NM within the SN in vivo. In this disclosure, ¹⁸F-P3BZA PET/MRI could be used to visualize changes of NM and iron at the same time in vivo.

PET/MRI is a hybrid imaging technology that incorporates PET functional imaging and MRI soft tissue morphological imaging²³.

In this disclosure, we have shown that ¹⁸F-P3BZA detect and bind melanin specifically in vitro and in vivo (PET). As is shown in example 1, the correlation analysis demonstrates strong correlation between the uptake of ¹⁸F-P3BZA and melanin content in pRPE cells in vitro (R²=0.91, FIG. 2C). In our previous study²⁴, we found that ¹⁸F-P₃BZA quickly accumulated in both melanotic cell lines, MCF-7-TYR and B16F10, and reached 5.57±0.31% and 6.85±0.08% at 0.5 h incubation time, respectively. The uptake value continued to increase at 1 h, with a value of 7.14±0.18% for MCF-7-TYR and 8.55±0.24% for B16F10. In comparison, for control MCF-7 cells no significant accumulation of ¹⁸F-P3BZA was observed and the uptake values were only 0.25±0.05% and 0.32±0.07% at 0.5 and 1 h, respectively. The actually proved melanin-binding feature of ¹⁸F-P3BZA provides us vast potential to diagnose early PD by detecting the NM content in SN using PET.

MRI is a powerful tool for PD diagnosis. This diagnose methods including the following 3 categories of SN biomarkers: (1) structural markers, which always combine with nueromelanin or iron sensitive sequences, can be used to visualize or segment the SN; (2) diffusion imaging and can be used to differentiate and diagnose early PD and to monitor disease progression; (3) functional markers, such as resting-state fMRI, which can be used to investigate functional changes and pathophysiological correlates²⁵.

Structural imaging and magnetization transfer focus on SN volume²⁶. The SN is comprised with the SN pars compacta (SNc) and the SN pars reticulata (SNr). The SNc neurons project to the striatum, globus pallidus, subthalamic nucleus, anterior thalamic nuclei, and prefrontal cortex. While the SNr neurons project to the ventral thalamic nuclei and prefrontal cortex and receive afferents from the striatum, external globus pallidus, and subthalamic nucleus. Loss of dopaminergic cells predominates in the ventrolateral SNc, other than in SNr, and is mainly responsible for PD genesis. Magnetization transfer (MT) imaging could provide better contrast and more accurate delineation of the SNc from SNr compare to conventional MRI. In a clinical trial, SN volume was detected to reduce around 15% in PD patients using MT^{25,27}.

However, conventional MRI has most often been found to be negative in PD (especially early stage PD) or to show nonspecific findings because the intrinsic weakness of MRI-low sensitivity²⁸.

For melanin-sensitive imaging, the biomarker is nueromelanin and iron²⁹. Neuromelanin can induce paramagnetic T1-shortening when combined with metals such as iron and copper, which appear as areas of high signal intensity. Using 3-Tesla MRI, Parkinson's disease (PD) demonstrated as the reduction of the neuromelanin-positive volume of substantia nigra (SN) pars compacta (SNc). Fast spin-echo (FSE) T1-weighted sequence was optimized to detect neuromelanin-related contrast (NRC)³⁰.

Iron is a powerful catalyst of biologic oxidation, which involved in neurodegeneration in PD. Post-mortem studies found that iron levels in SN increased significantly with PD compared to age-matched normal control brains. Most of iron in SN tissue is stored in the form of ferritin iron, which is known to increase on transverse relaxation rates (R2, the reciprocal of transverse relaxation time) in a magnetic field-dependent manner on conventional structural magnetic resonance (MR) imaging. MRI can measure iron level indirectly. In PD patients, it is chartered as decreased relaxation times and increased relaxation rates (R2/R2*) in the SN³¹.

Advanced MR techniques, such as diffusion-weighted imaging (DWI), diffusion tensor imaging (DTI), proton magnetic resonance spectroscopy CH MRS) and resting-state functional connectivity may provide biomarkers of SN degeneration. These markers include reduced T2 and T2* relaxation times, reduced fractional anisotropy and reduced probability of connection between the SN and the striatum and thalamus. DWI could improve contrast between SN and surrounding tissue and reported reduced SNc width in PD. While by DTI, we can investigate the nigrostriatal and nigrothalamic fiber tracks^{32,33}.

Above all, neuromelanin and iron represent the characteristic changes, are closely related to the pathogenesis of PD (including early PD), and can be considered as sensitive surrogate for neuroimaging^{3,34}. It is confirmed by a lot of studies that MRI, despite all of its failings such as low sensitivity, is a powerful tool for PD diagnosis. Our present research indicates that ¹⁸F-P3BZA is a potent melanin-targeting probe. When combine with PET, the most sensitive imaging technique, could compensate the shortcoming of MRI effectively. PET is very sensitive at detecting and quantifying a low abundance molecular target on or within diseased cells. MRI is capable of providing exquisite anatomy as well as physiological measurements of disease states. Thus, the two modalities provide complimentary information. ¹⁸F-P3BZA PET/MRI could provides unprecedented opportunities to visualize, detect diagnose and monitor PD in both early and advanced stage in vivo.

### REFERENCES

1. Zecca, L., Zucca, F. A., Albertini, A., Rizzio, E. & Fariello, R. G. A proposed dual role of neuromelanin in the pathogenesis of Parkinson's disease. Neurology 67, S8-11 (2006).
2. Worth, P. F. How to treat Parkinson's disease in 2013. Clin Med 13, 93-96 (2013).
3. Pagan, F. L. Improving outcomes through early diagnosis of Parkinson's disease. The American journal of managed care 18, S176-182 (2012).
4. Godau, J., Hussl, A., Lolekha, P., Stoessl, A. J. & Seppi, K. Neuroimaging: current role in detecting pre-motor Parkinson's disease. Movement disorders: official journal of the Movement Disorder Society 27, 634-643 (2012).
5. Betarbet, R., Sherer, T. B. & Greenamyre, J. T. Animal models of Parkinson's disease. BioEssays: news and reviews in molecular, cellular and developmental biology 24, 308-318 (2002).
6. Benadiba, M., Luurtsema, G., Wichert-Ana, L., Buchpigel, C. A. & Busatto Filho, G. New molecular targets for PET and SPECT imaging in neurodegenerative diseases. Rev Bras Psiquiatr 34 Suppl 2, S125-136 (2012).
7. Seibyl, J., Russell, D., Jennings, D. & Marek, K. The molecular basis of dopaminergic brain imaging in Parkinson's disease. Q J Nucl Med Mol Imaging 56, 4-16 (2012).
8. Tatsch, K. Positron emission tomography in diagnosis and differential diagnosis of Parkinson's disease. Neuro-degenerative diseases 7, 330-340 (2010).
9. Kaasinen, V., et al. Upregulation of putaminal dopamine D2 receptors in early Parkinson's disease: a comparative PET study with [11C] raclopride and [11C]N-methylspiperone. Journal of nuclear medicine: official publication, Society of Nuclear Medicine 41, 65-70 (2000).
10. Thobois, S., Guillouet, S. & Broussolle, E. Contributions of PET and SPECT to the understanding of the pathophysiology of Parkinson's disease. Neurophysiologie clinique=Clinical neurophysiology 31, 321-340 (2001).
11. Bruck, A., Aalto, S., Nurmi, E., Bergman, J. & Rinne, J. O. Cortical 6-[18F]fluoro-L-dopa uptake and frontal cognitive functions in early Parkinson's disease. Neurobiology of aging 26, 891-898 (2005).
12. Bruck, A., et al. Striatal subregional 6-[18F]fluoro-L-dopa uptake in early Parkinson's disease: a two-year follow-up study. Movement disorders: official journal of the Movement Disorder Society 21, 958-963 (2006).
13. Giza, E., Gotzamani-Psarrakou, A. & Bostantjopoulou, S. Imaging beyond the striatonigral dopaminergic system in Parkinson's disease. Hellenic journal of nuclear medicine 15, 224-232 (2012).
14. Benamer, H. T., et al. Prospective study of presynaptic dopaminergic imaging in patients with mild parkinsonism and tremor disorders: part 1. Baseline and 3-month observations. Movement disorders: official journal of the Movement Disorder Society 18, 977-984 (2003).
15. Bohnen, N. I. & Albin, R. L. The cholinergic system and Parkinson disease. Behavioural brain research 221, 564-573 (2011).
16. Kuhl, D. E., et al. In vivo mapping of cholinergic terminals in normal aging, Alzheimer's disease, and Parkinson's disease. Annals of neurology 40, 399-410 (1996).
17. Kikuchi, A., et al. In vivo visualization of alpha-synuclein deposition by carbon-11-labelled 2-[2-(2-dimethylaminothiazol-5-yl)ethenyl]-6-[2-(fluoro)ethoxy]benzoxazole positron emission tomography in multiple system atrophy. Brain: a journal of neurology 133, 1772-1778 (2010).
18. Versijpt, J. J., et al. Assessment of neuroinflammation and microglial activation in Alzheimer's disease with radiolabelled PK11195 and single photon emission computed tomography. A pilot study. European neurology 50, 39-47 (2003).
19. Eidelberg, D., et al. The metabolic topography of parkinsonism. Journal of cerebral blood flow and metabolism: official journal of the International Society of Cerebral Blood Flow and Metabolism 14, 783-801 (1994).
20. Silva, M. D., et al. Regional, kinetic [(18)F]FDG PET imaging of a unilateral Parkinsonian animal model. American journal of nuclear medicine and molecular imaging 3, 129-141 (2013).
21. Zhang, W., et al. Human neuromelanin: an endogenous microglial activator for dopaminergic neuron death. Front Biosci (Elite Ed) 5, 1-11 (2013).
22. Bezard, E., et al. Relationship between the appearance of symptoms and the level of nigrostriatal degeneration in a progressive 1-methyl-4-phenyl-1,2,3,6-tetrahydropyridine-lesioned macaque model of Parkinson's disease. The Journal of neuroscience: the official journal of the Society for Neuroscience 21, 6853-6861 (2001).
23. Catana, C., Drzezga, A., Heiss, W. D. & Rosen, B. R. PET/MRI for neurologic applications. Journal of nuclear medicine: official publication, Society of Nuclear Medicine 53, 1916-1925 (2012).
24. Liu, H., et al. Development of 18F-labeled picolinamide probes for PET imaging of malignant melanoma. Journal of medicinal chemistry 56, 895-901 (2013).
25. Lehericy, S., Sharman, M. A., Dos Santos, C. L., Paquin, R. & Gallea, C. Magnetic resonance imaging of the substantia nigra in Parkinson's disease. Movement disorders: official journal of the Movement Disorder Society 27, 822-830 (2012).
26. Ziegler, D. A. & Augustinack, J. C. Harnessing advances in structural MRI to enhance research on Parkinson's disease. Imaging in medicine 5, 91-94 (2013).
27. Ogisu, K., et al. 3D neuromelanin-sensitive magnetic resonance imaging with semi-automated volume measurement of the substantia nigra pars compacta for diagnosis of Parkinson's disease. Neuroradiology (2013).
28. Morgen, K., et al. Structural brain abnormalities in patients with Parkinson disease: a comparative voxel-based analysis using T1-weighted MR imaging and magnetization transfer imaging. AJNR. American journal of neuroradiology 32, 2080-2086 (2011).
29. Schwarz, S. T., et al. T1-weighted MRI shows stage-dependent substantia nigra signal loss in Parkinson's disease. Movement disorders: official journal of the Movement Disorder Society 26, 1633-1638(2011).
30. Kitao, S., et al. Correlation between pathology and neuromelanin MR imaging in Parkinson's disease and dementia with Lewy bodies. Neuroradiology (2013).
31. Mahlknecht, P., et al. Significance of MRI in diagnosis and differential diagnosis of Parkinson's disease. Neuro-degenerative diseases 7, 300-318 (2010).
32. Schocke, M. F., et al. Diffusion-weighted MRI differentiates the Parkinson variant of multiple system atrophy from PD. Neurology 58, 575-580 (2002).
33. Cochrane, C. J. & Ebmeier, K. P. Diffusion tensor imaging in parkinsonian syndromes: a systematic review and meta-analysis. Neurology 80, 857-864 (2013).
34. Thobois, S., Ballanger, B., Poisson, A. & Broussolle, E. [Imaging non motor signs in Parkinson's disease]. Revue neurologique 168, 576-584 (2012).

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range. In an embodiment, the term “about” can include traditional rounding according to significant figures of the numerical value. In addition, the phrase “about ‘x’ to ‘y’” includes “about ‘x’ to about ‘y’”.

It should be emphasized that the above-described embodiments of the present disclosure are merely possible examples of implementations, and are merely set forth for a clear understanding of the principles of this disclosure. Many variations and modifications may be made to the above-described embodiment(s) of the disclosure without departing substantially from the spirit and principles of the disclosure. All such modifications and variations are intended to be included herein within the scope of this disclosure and protected by the following claims.

We claim at least the following:

1. A method of diagnosing the presence of a Parkinson's disease in a subject comprising:
administering to the subject a labeled probe, wherein the labeled probe has an affinity for neuromelanin present in the brain, wherein the amount of neuromelanin present in the brain is a biomarker for Parkinson's disease;
detecting the labeled probe, wherein the amount of the labeled probe present in the brain corresponds with the amount of neuromelanin present in the brain,
wherein the labeled probe has the following structure: 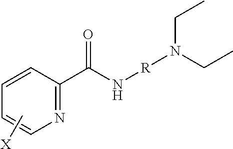
wherein X is selected from the group consisting of: ¹⁸F, ¹¹C, ¹²⁵I, ¹²⁴I, ¹³¹I, ¹²³I, ³²Cl, ³³Cl, ³⁴Cl, ⁶⁸Ga, ⁷⁴Br, ⁷⁵Br, ⁷⁶Br, ⁷⁷Br, ⁷⁸Br, ⁸⁹Zr, ¹⁸⁶Re, ¹⁸⁸Re, ⁹⁰Y, ⁸⁶Y, ¹⁷⁷Lu, and ¹⁵³Sm,
wherein R is selected from the group consisting of: a substituted or unsubstituted, linear or branched, CₙH₂ₙ, where n is 1 to 12, and a substituted or unsubstituted, linear or branched, alkene group having 2 to 12 carbons, and
wherein X is positioned on anyone of the carbon atoms on the heteroaromatic ring.

2. The method of claim 1, wherein X is ¹⁸F.

3. The method of claim 1, further comprising: imaging at least a portion of the brain of the subject.

4. A method of monitoring the progress of a Parkinson's disease in a subject comprising:
administering to the subject a labeled probe, wherein the labeled probe has an affinity for neuromelanin present in the brain, wherein the amount of neuromelanin present in the brain is a biomarker for Parkinson's disease;
detecting the labeled probe, wherein the amount of the labeled probe present in the brain corresponds to the amount of neuromelanin present in the brain; and
repeating the detection of the labeled probe periodically to determine the change in the amount of neuromelanin present in the brain, wherein a change is related to the progression or regression of Parkinson's disease,
wherein the labeled probe has the following structure: 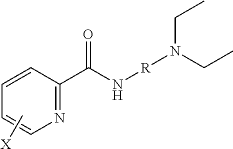
wherein X is selected from the group consisting of: ¹⁸F, ¹¹C, ¹²⁵I, ¹²⁴I, ¹³¹I, ¹²³I, ³²Cl, ³³Cl, ³⁴Cl, ⁶⁸Ga, ⁷⁴Br, ⁷⁵Br, ⁷⁶Br, ⁷⁷Br, ⁷⁸Br, ⁸⁹Zr, ¹⁸⁶Re, ¹⁸⁸Re, ⁹⁰Y, ⁸⁶Y, ¹⁷⁷Lu, and ¹⁵³Sm, and R is selected from the group consisting of: a substituted or unsubstituted, linear or branched, CₙH₂ₙ, where n is 1 to 12, and a substituted or unsubstituted, linear or branched, an alkene group having 2 to 12 carbons, wherein X is positioned on anyone of the carbon atoms on the heteroaromatic ring.

5. The method of claim 4, further comprising: imaging at least a portion of the subject;

6. The method of claim 5, further comprising: repeating the steps of detecting, imaging, or both periodically to monitor the dimensions of the location corresponding to the retinal pigment epithelial cells.

7. The method of claim 6, wherein X is ¹⁸F.

8. A method of diagnosing the presence of a Parkinson's disease in a subject comprising:
administering to the subject a labeled probe, wherein the subject has been administered retinal pigment epithelial cells, wherein the labeled probe has an affinity for retinal pigment epithelial cells; and
detecting the labeled probe, wherein the location of the labeled probe corresponds to the location of the retinal pigment epithelial cells,
wherein the labeled probe has the following structure: 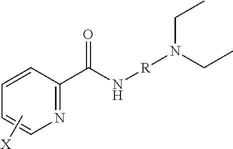
wherein X is selected from the group consisting of: ¹⁸F, ¹¹C, ¹²⁵I, ¹²⁴I, ¹³¹I, ¹²³I, ³²Cl, ³³Cl, ³⁴Cl, ⁶⁸Ga, ⁷⁴Br, ⁷⁵Br, ⁷⁶Br, ⁷⁷Br, ⁷⁸Br, ⁸⁹Zr, ¹⁸⁶Re, ¹⁸⁸Re, ⁹⁰Y, ⁸⁶Y, ¹⁷⁷Lu, and ¹⁵³Sm,
wherein R is selected from the group consisting of: a substituted or unsubstituted, linear or branched, CₙH₂ₙ, where n is 1 to 12, and a substituted or unsubstituted, linear or branched, alkene group having 2 to 12 carbons, and
wherein X is positioned on anyone of the carbon atoms on the heteroaromatic ring.

9. The method of claim 8, wherein X is ¹⁸F.

10. The method of claim 8, further comprising: imaging at least a portion of the subject.

11. A method of monitoring the progress of a Parkinson's disease in a subject comprising:
administering to the subject a labeled probe, wherein the subject has been administered retinal pigment epithelial cells, wherein the labeled probe has an affinity for retinal pigment epithelial cells;
detecting the labeled probe, wherein the location of the labeled probe corresponds to the location of the retinal pigment epithelial cells, wherein the dimensions of the location is monitored over time,
wherein the labeled probe has the following structure: 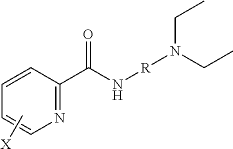
wherein X is selected from the group consisting of: ¹⁸F, ¹¹C, ¹²⁵I, ¹²⁴I, ¹³¹I, ¹²³I, ³²Cl, ³³Cl, ³⁴Cl, ⁶⁸Ga, ⁷⁴Br, ⁷⁵Br, ⁷⁶Br, ⁷⁷Br, ⁷⁸Br, ⁸⁹Zr, ¹⁸⁶Re, ¹⁸⁸Re, ⁹⁰Y, ⁸⁶Y, ¹⁷⁷Lu, and ¹⁵³Sm,
wherein R is selected from the group consisting of: a substituted or unsubstituted, linear or branched, CₙH₂ₙ, where n is 1 to 12, and a substituted or unsubstituted, linear or branched, alkene group having 2 to 12 carbons, and
wherein X is positioned on anyone of the carbon atoms on the heteroaromatic ring.

12. The method of claim 11, further comprising imaging at least a portion of the subject.

13. The method of claim 12, further comprising repeating the steps of detecting, imaging, or both periodically to monitor the dimensions of the location corresponding to the retinal pigment epithelial cells.

14. The method of claim 11, wherein X is ¹⁸F.
